(19) United States
(12) Patent Application Publication
(10) Pub. No.: US 2026/0354411 A1
(43) Pub. Date: Nov. 12, 2026

(54) STRAINS OF LEUCONOSTOC CITREUM, THEIR USE FOR PRODUCING FERMENTED VEGAN DESSERTS, ESPECIALLY SUCH COMPRISING FERMENTED PEA PROTEINS AND VEGAN DESSERTS COMPRISING FERMENTED PEA PROTEIN AND SAID STRAINS

(71) Applicant: Cargill, Incorporated, Wayzata, MN (US)

(72) Inventor: Vasileios POTHAKOS, Brussels (BE)

(73) Assignee: Cargill, Incorporated, Wayzata, MN (US)

(21) Appl. No.: 19/124,009

(22) PCT Filed: Nov. 8, 2023
(86) PCT No.: PCT/US23/79037

§ 371 Date: Apr. 24, 2025

(30) Foreign Application Priority Data
Nov. 8, 2022 (EP) 22206229.1

(51) Int. Cl. A23J 3/14; A23L 9/10; A23L 11/50; A23L 29/30; C12N 1/205; C12R 1/01

(52) U.S. Cl. CPC A23J 3/14; A23L 9/12; A23L 11/50; A23L 29/30; C12N 1/205; A23V 2400/315; C12R 2001/01

(57) ABSTRACT

Described herein are composition and methods for the production of a vegan dessert composition including a pea protein fermentate. An initial composition including a pea protein, a Leuconostoc citreum bacterium, and sucrose may be fermented for a time and under conditions suitable to produce a pea protein fermentate. The resulting pea protein fermentate will have increased viscosity, improved aspect, and improved sensory attributes relative to an equivalent pea protein composition that has not been contacted with or fermented by a Leuconostoc citreum bacterium. The pea protein fermentate is incorporated into a vegan dessert composition.

### CROSS-REFERENCE TO RELATED APPLICATIONS

This application claims the benefit of European Priority Application No. 22206229.1, filed Nov. 8, 2022, which is incorporated by reference herein in its entirety.

### BACKGROUND

As public interest in plant-based proteins continues to grow, pea proteins are being used in more applications across the food and beverage industry. For example, pea proteins can be found in many commercially available energy bars, meal-replacement shakes, plat-based meat alternatives, breakfast cereal products, supplement products, and the like. While there are many commercially available sources of pea protein and many commercially available products containing pea proteins, there are opportunities to improve not only the flavor and sensory attributes of pea proteins but also to improve the functional performance of the pea protein.

Fermentation is an antient and widely used process to change flavor and functional properties of food. For example, the fermentation of cabbage can produce sauerkraut and kimchi products, fermentation of milk can produce cheese and yogurt, and fermentation of fruits, sugars, and cereal grains can produce alcoholic beverages. However, the practice of fermentation still has many broad applications and potentials that have yet to discovered and developed.

Described herein are compositions and methods for the fermentation of pea proteins resulting in beneficial improvements in both sensory aspects and functional characteristics.

### SUMMARY

The present disclosure provides a vegan dessert composition comprising a fermented pea protein composition; wherein the fermented pea protein composition is made by contacting a pea protein with Leuconostoc citreum B3K7 or Leuconostoc citreum C22B11 in the presence of sucrose; and optionally, a sweetener, a flavoring, or combinations thereof. The vegan dessert composition may be free of added starch. The vegan dessert composition may comprise a sweetener comprising steviol glycosides, mogrosides, sucrose, fructose, glucose, erythritol, maltitol, lactitol, sorbitol, mannitol, xylitol, sucralose, acesulfame K, aspartame, saccharin, or combinations thereof.

The disclosure also provides a method for producing a vegan dessert composition, the method comprising: (i) contacting a pea protein composition comprising a pea protein and sucrose with Leuconostoc citreum B3K7 or Leuconostoc citreum C22B11 for a time and under conditions sufficient to produce a fermented pea protein product; (ii) optionally, inactivating the Leuconostoc citreum bacteria in the fermented pea protein (e.g., by pasteurization, heat killing, irradiation, or chemical treatment) and (iii) adding to the fermented pea protein product a sweetener, a flavoring, or combinations thereof, whereby the vegan pudding composition is produced. The pea protein composition may comprise between 1 wt % and 20 wt %, 2 wt % and 18 wt %, or 4 wt % and 15 wt % pea protein and/or between 1 wt % and 30 wt %, 2 wt % and 25 wt %, or 5 wt % and 20 wt % sucrose. The pea protein composition may be fermented for at least 6, at least 12, at least 18, or at least 24 hours; and/or the pea protein composition is fermented at a temperature between 20 °C and 30 °C, between 22 °C and 28 °C, between 24 °C and 26 °C, or about 25 °C. The method may additionally comprise the step of stirring the contacted pea protein composition during or after step (i). The sweetener may comprise steviol glycosides, mogrosides, sucrose, fructose, glucose, erythritol, maltitol, lactitol, sorbitol, mannitol, xylitol, sucralose, acesulfame K, aspartame, saccharin, or combinations thereof.

The disclosure also provides for an isolated Leuconostoc citreum B3K7 bacterial cell and an isolated Leuconostoc citreum C22B11 bacterial cell.

The disclosure also provides for use of a Leuconostoc citreum B3K7 bacteria cell and/or a Leuconostoc citreum C22B11 bacterial cell for the production of a vegan dessert composition.

The disclosure also provides a composition comprising a fermented pea protein, a non-viable Leuconostoc citreum B3K7 and/or Leuconostoc citreum C22B11 bacterium; wherein the composition is free of added sucrose and/or wherein the composition is free of added starch.

### BRIEF DESCRIPTION OF THE FIGURES

This patent or application contains at least one drawing executed in color. Copies of this patent or patent application publication with color drawings will be provided by the Office upon request and the payment of the necessary fee.

The drawings illustrate generally, by way of example, but not by way of limitation, various aspects discussed herein.

FIG. 1 shows photos of the visual aspect of fermentate samples 1.1-1.8, before and after stirring, as outlined in Example 1.

FIG. 2 shows photos of the visual aspect of fermentate samples 1.9-1.16, before and after stirring, as outlined in Example 1.

FIG. 3 shows photos of the visual aspect of fermentate samples 1.17-1.24, before and after stirring, as outlined in Example 1.

FIG. 4 shows stills from a video comparing the aspect of samples 1.17 and 1.24.

FIG. 5 shows viscosity profiles of the Leuconostoc citreum B3K7 fermentates of samples 1.1-1.8.

FIG. 6 shows viscosity profiles of the Leuconostoc citreum C22B11 fermentates of samples 1.9-1.16.

FIG. 7 shows viscosity profiles of the Leuconostoc citreum C18X24 fermentates of samples 1.17-1.24.

FIG. 8 shows the reduction in syneresis in sample 1.2 relative to a control starch and protein suspension sample.

FIG. 9 shows a comparison between the aspect of a reference vegan dessert formulation made with pea protein, starch, and carrageenan and the aspect of samples 1.2 and 1.10.

FIG. 10 shows the aspect and consistency of a reference vegan dessert formulation made with pea protein, starch, and carrageenan along side samples 1.2, 1.10, and 1.18.

FIG. 11 shows relative changes in sensory attributes when sample 1.2 is compared to the reference vegan dessert formulation outlined in Example 4.

FIG. 12 shows viscosity of samples outlined in Example 5.

FIG. 13 shows viscosity profiles of Protocol A samples from Example 5.

FIG. 14 shows viscosity profiles of Protocol B samples from Example 5.

FIG. 15 shows viscosity profiles of Protocol C samples from Example 5.

FIG. 16 shows residual sucrose concentration of the samples outlined in Example 5.

FIG. 17 shows viscosity of samples outlined in Example 6.

FIG. 18 shows viscosity profiles of 4 wt % pea protein fermentation samples from Example 6.

FIG. 19 shows viscosity profiles of 15 wt % pea protein fermentation samples from Example 6.

FIG. 20 shows viscosity profiles of vital wheat gluten fermentation samples from Example 6.

FIG. 21 shows viscosity profiles of corn protein fermentation samples from Example 6.

### DETAILED DESCRIPTION

Reference will now be made in detail to certain aspects of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

In this document, the terms “a,” “an,” or “the” are used to include one or more than one unless the context clearly dictates otherwise. The term “or” is used to refer to a nonexclusive “or” unless otherwise indicated. All publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

Unless expressly stated, ppm (parts per million), percentage, and ratios are on a by weight basis. Percentage on a by weight basis is also referred to as wt % or % (wt) below.

This disclosure relates to compositions and methods for the production of pea protein fermentates. As described herein, the fermented pea protein product is characterized by increased viscosity, altered visual appearance, and/or alterations of one or more sensory attributes relative to the protein prior to fermentation. In general, the pea protein is fermented with a Leuconostoc citreum bacterium.

### Fermentates

This disclosure relates to compositions comprising a pea protein, a Leuconostoc citreum bacterium, and sucrose, as well as methods for use of said composition in the production of a fermented pea protein product.

In general, the starting composition will include a pea protein (i.e., protein extracted and/or derived from seed or pod fruit of Pisum sativun). The pea protein may be from any suitable source. The pea protein may be a pea protein isolate, a pea protein concentrate, or combinations thereof. Suitable pea proteins are available commercially and may include, but are not limited to, NUTRALYS® (Roquette), PISANE® Pea Protein (COSUCRA™), RADIPURE™ pea protein isolate, and Pea Protein 870 (PURIS®). Suitable pea protein compositions may include at least 50%, at least 60%, at least 70%, at least 75%, or at least 80% protein. The starting composition may include between 1 wt % and 20 wt %, 2 wt % and 18 wt %, or 4 wt % and 15 wt % pea protein. For example, the starting composition may include equal to or about 1, 2, 3, 4, 5, 6, 7, 8, 9, 10, 11, 12, 13, 14, 15, 16, 17, 18, 19, or 20 wt % pea protein.

The starting composition additionally includes sucrose. The composition may include between 1 wt % and 30 wt %, 2 wt % and 25 wt %, or 5 wt % and 20 wt % sucrose, e.g., equal to or about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 wt % sucrose. The sucrose may be from any suitable source. One skilled in the art will recognize suitable sources, including commercially available sources, or sucrose.

The staring composition may include the pea protein and the sucrose in a ratio by weight between about 3:1 to 1:10, about 2:1 to 1:8, or about 1:1 to 1:4, preferable between about 1:1 to 1:4.

The starting composition additionally includes a Leuconostoc citreum or Leuconostoc pseudomesenteroides bacterium. The Leuconostoc citreum or Leuconostoc pseudomesenteroides bacteria may be from any suitable source. Suitable Leuconostoc citreum or Leuconostoc pseudomesenteroides bacterium include, but are not limited to, L. citreum strain B3K7 (deposited with the Belgian Coordinated Collections of Micro-organisms (BCCM) Laboratorium voor Microbiologie—Bacterienverzameling (LMG), Ghent University K. L. Ledeganckstraat 35, 9000 Gent, Belgium, on Sep. 27, 2022 under the accession number LMG P-32801), L. citreum strain C22B11 (deposited with the BCCM/LMG, Ghent University K. L. Ledeganckstraat 35, 9000 Gent, Belgium, on Sep. 27, 2022 under the accession number LMG P-32800), L. pseudomesenteroides strain C18X24 (deposited with the BCCM/LMG, Ghent University K. L. Ledeganckstraat 35, 9000 Gent, Belgium, on Jun. 21, 2023 under the accession number LMG P-33195), L. citreum strain C18X1 (deposited with the BCCM/LMG, Ghent University K. L. Ledeganckstraat 35, 9000 Gent, Belgium, on Oct. 20, 2022 under the accession number LMG P-32799), and combinations thereof.

In priority application EP 22206229.1, filed Nov. 8, 2022, strain C18X24 was mistakenly indicated as strain C18X1 in the examples and throughout the specification, drawings, and claims. Appropriate correction is made herein. Accordingly, data using strain C18X24 was presented in application EP 22206070.9 and strain C18X24 was fully supported as of the filing date of the priority application, Nov. 8, 2022. Additional data is presented herein based on strain C18X1 and this strain is distinct from the mislabeled strain in the priority application.

The staring composition is fermented for a time and under conditions sufficient to produce a pea protein fermentate. For example, the pea protein may be contacted with the L. citreum bacterium in the presence of sucrose at a temperature between 20 °C and 30 °C, between 22 °C and 28 °C, between 24 °C and 26 °C, or about 25 °C for at least 6, at least 12, at least 18, or at least 24 hours.

Herein “fermented pea protein product” and “pea protein fermentate” are used interchangeably and refer to a composition produced by microbial fermentation of a pea protein and includes (i) said pea protein; (ii) metabolites produced by the microorganisms during fermentation of the pea protein; (iii) non-viable microorganisms used in the fermentation process; and (iv) water. The pea protein fermentate may include metabolites such as, but not limited to, fructose, alpha-glucan, polyols, organic acids, and combinations thereof. For example, the pea protein fermentate may include between 1 wt % and 20 wt %, between 2 wt % and 15 wt %, or between 5 wt % and 10 wt % alpha-glucan. The pea protein fermentate may include between 1 wt % and 20 wt %, between 2 wt % and 15 wt %, or between 5 wt % and 10 wt % fructose. The pea protein fermentate may include between 0.1 wt % and 10 wt %, between 0.5% and 8%, or between 1 wt % and 5 wt % polyols. The pea protein fermentate may include between 0.1 wt % and 10 wt %, between 0.5% and 8%, or between 1 wt % and 5 wt % organic acids. The pea protein fermentate may include between 0.01 wt % and 5 wt %, between 0.05 wt % and 2 wt %, or between 0.1 wt % and 1 wt % dietary fiber. In an example, the pea protein fermentate may include between 5 wt % and 10 wt % fructose, between 5 wt % and 10 wt % alpha-glucan, between 1 wt % and 5 wt % polyols, between 1 wt % and 5 wt % organic acids, between 0.1 wt % and 1 wt % dietary fiber, protein, fat, and water.

The pea protein fermentate may include an alpha-glucan that is a linear alpha-glucan. In general, the alpha-glucan may have an average molecular weight of at least 300 kDa, at least 500 kDa, at least 750 kDa, at least 1 MDa, at least 2 MDa, at least 3 MDa, at least 4 MDa, at least 5 MDa, at least 6 MDa, at least 7 MDa, at least 8 MDa, or about 9 MDa. The alpha-glucan may have an average molecular weight between 300 kDa and 9 MDa.

The pea protein fermentate may be processed using an inactivation step, in which microorganisms are rendered non-viable. For example, the pea protein fermentate may be pasteurized, heat killed, irradiated, or chemically treated to make any remaining microorganisms non-viable. The pea protein fermentate may additionally or alternatively undergo physical methods by which the microorganisms are separated, for example, by filtration.

Although sucrose is used to produce the pea protein fermentate, the resulting pea protein fermentate may be free of sucrose. In other words, all of the sucrose present in the initial starting composition may be utilized by the L. citreum during the fermentation such that the resulting pea protein fermentate is free of sucrose. In an example, the pea protein fermentate may include between 5 wt % and 10 wt % fructose, between 5 wt % and 10 wt % alpha-glucan, between 1 wt % and 5 wt % polyols, between 1 wt % and 5 wt % organic acids, between 0.1 wt % and 1 wt % dietary fiber, protein, fat, and water and is free of sucrose (e.g., less than 1 wt %, less than 0.5 wt %, less than 0.1 wt %, less than 0.01 wt %, or less than the detection level of sucrose). Likewise, the pea protein fermentate may be free of added sucrose, whereby all of the sucrose in the starting composition is used up, and no additional sucrose is added to the produced pea protein fermentate.

The pea protein fermentate may be free of added starch. As used herein, “free of added starch” refers to a composition in which no starch ingredient has been added but may include starch produced as a result of a fermentation process or reaction. For example, the pea protein fermentate may include starch produced by the microorganism during fermentation but is free from the addition of any other starch ingredient component.

Additionally, the pea protein fermentate may be stirred to form a stirred pea protein fermentate. The pea protein fermentate may be stirred manually or mechanically. The pea protein fermentate may be stirred for at least 2, 5, 10, 15, 30, 45 or 60 seconds, and/or until the desired texture is achieved.

The pea protein fermentate may have a pH between about 4 and 5, between 4.1 and 4.8, or between 4.2 and 4.7. In general, higher concentrations of sucrose in the starting composition will produce pea protein fermentate with slightly high pH values.

In general, pea protein fermentates described herein are characterized by an increased viscosity relative to an equivalent pea protein composition that has not been contacted with/fermented using a L. citreum bacterium. The viscosity of the pea protein fermentate may be at least 700, 800, 900, 1000, 1100, 1200, 1300, 1400, 1500, 1800, 2000, 2200, 2500, 2800, 3000, 3200, 3500, 3800, or at least 4000 cP when measured at after 5 minutes of stirring at 250 rpm and 25 °C. In general, higher concentrations of pea protein in the starting composition will produce pea protein fermentates with higher viscosities. Likewise, higher concentrations of sucrose in the starting composition will produce pea protein fermentates with higher viscosities. Therefore, as is apparent from the data presented herein, one of skill in the art can tailor the starting composition, altering both the pea protein and sucrose concentrations, to result in a pea protein fermentate with a specific desired viscosity.

Pea protein fermentates described herein are characterized by an altered visual and physical appearance relative to an equivalent pea protein composition that has not been contacted with/fermented using a L. citreum bacterium. For example, the pea protein fermentates may be more glossy, more shiny, gooier, and/or have a more gel-like aspect than an equivalent pea protein composition that has not been contacted with/fermented using a L. citreum bacterium. A “glossy” aspect of the pea protein fermentate is one in which the pea protein fermentate is smooth (i.e., is not chunky or grainy in appearance) and is shiny. A “shiny” aspect is one in which the surface of the pea protein fermentate reflects light. A “gooey” aspect is one in which the pea protein fermentate appears soft and sticky. A “gel-like” aspect is one in which the pea protein fermentate appears thick, slightly stick, and somewhat solid/firm. Evaluation of the aspect of the pea protein fermentate may be done with the naked eye or may be aided by measuring one of the aforementioned traits mechanically. Aspect evaluation may be aided by stirring or disrupting the pea protein fermentate (e.g., with a spoon) to see how the texture and appearances effect the aspect.

### Sensory Attributes

The compositions and methods described herein are characterized by modulation of one or more sensory attributes relative to an equivalent pea protein composition that has not been contacted with/fermented using a L. citreum bacterium. Modulated sensory attributes may include, but are not limited to, bitterness, sourness, sweetness, pea flavor, green/grassy notes, nutty notes, chalky flavor, acidic notes, and umami flavor.

For example, the pea protein fermentates described herein have increased sweetness, increased sourness, reduced pea flavor, reduced green/grassy notes, increased nutty notes, increased umami flavor, or combinations thereof relative to an equivalent pea protein composition that has not been contacted with/fermented using a L. citreum bacterium.

As used herein, “sensory attribute” refers to a taste, aroma, and or flavor associated with a given composition that has characteristic properties familiar to one trained in sensory evaluation. For example, a salty taste is associated with sodium chloride, a sweet taste is associated with sucrose, a sour taste is associated with citric acid, a bitter taste is associated with caffeine, and an umami taste is associated with monosodium glutamate (MSG).

As used herein, “taste” refers to sensory perception on the tongue. For example, the 5 basic tastes are sweet, sour, salty, bitter, and umami.

As used herein, “aroma” refers to the orthonasal perception in the nasal cavity.

As used herein, “flavor” refers to the taste and retronasal perception in the nasal cavity.

As used herein, “off-taste(s)” refer to a taste or flavor attribute profile that is not characteristic or usually associated with a substance or composition as described herein and/or a characteristic taste or flavor associated with a substance or composition that is undesirable. For example, the off-taste may be an undesirable taste such as bitterness, undesirable mouthfeel such as astringency, mouth drying, undesirable flavor such as rancid, cardboard, aftertaste, inconsistent flavor (e.g., a flavor with an uneven onset or intensity, a flavor that may be perceived too early or too late), and the like.

As used herein, “plant protein flavor” refers to the characteristic flavor(s) associated with and expected from plant-based proteins when said plant-based proteins are used as ingredients in food and beverage products. For example, plant protein flavors include beany, pea, corny, hay, green notes, barnyard, fermented, waxy, and combinations thereof that are usually found and expected from a plant-based protein. In general, certain characteristic plant protein flavors can be attributed to certain plant-based proteins. For example, pea proteins may be associated with green notes, pea flavor, and hay flavor; soy proteins may be associated with beany flavor and hay flavor, corn proteins may be associated with corny flavor and hay flavor, and potato proteins may be associated with barnyard flavor and fermented flavor.

A sensory panel can be used to determine the magnitude of, for example, reduction in bitterness or shifts in its temporal profile. Sensory panels are a scientific and reproducible method that is essential to the food and beverage industry. A sensory panel involves a group of two or more individual panelists. Panelists are instructed according to industry-recognized practices to avoid the influence of personal subjectivity and strengthen reproducibility. For example, panelists may objectively evaluate sensory attributes of a tested product but may not provide subjective attributes such as personal preference. In various aspects, the sensory panel can be conducted with two, three, four, five, six, or more panelists, in which the panelists identify and agree on a lexicon of sensory attributes for a given set of samples. After evaluating a specific sample, the panelists can assign a numerical intensity score for each attribute using an intensity scale. For example, intensity scales can range from 0 to 6 (i.e., 0=not detected, 1=trace, 2=slight, 3=moderate, 4=definite, 5=strong, 6=extreme), 0 to 9 (i.e., 0=not detected, 1=trace, 2=faint, 3=slight, 4=mild, 5=moderate, 6=definite, 7=strong, 8=very strong, 9=extreme), or 0 to 15, where 0 corresponds to the absence of the attribute, while 6, 9, or 15, respectively, corresponds to the upper bound extreme occurrence of the attribute. The panel may use a roundtable consensus approach, or the panelists may score and evaluate the sensory attribute(s) individually. Either format can further involve a panel leader who directs the discussion regarding terminology and directs the panel to evaluate particular products and attributes. In other aspects, a trained sensory panel can be utilized to assess specific attributes using descriptive analysis or time intensity methodologies.

As used herein, “panelist” refers to a highly trained expert taster, such as those commonly used for sensory methodologies such as descriptive analysis, and/or an experienced taster familiar with the sensory attribute(s) being tested. In some aspects, the panelist may be a trained panelist. A trained panelist has undergone training to understand the terms and sensory phenomenon associated with those sensory attributes relevant to the tested product and are aligned on the use of common descriptors for those sensory attributes of interest (i.e., a sensory lexicon). For example, a trained panelist testing a given composition will understand the terms and sensory attributes associated with said composition, e.g. saltiness, sourness, bitterness, astringency, mouthfeel, acidity, and the like. The trained panelist will have been trained against reference samples corresponding to the sensory attributes being tested and thus have calibrated to recognize and quantitatively assess such criteria. In some aspects, the panelist may be an experienced taster.

As used herein, “roundtable consensus approach” refers to the sensory panel assay methodology wherein panelists discus sensory attributes and intensities before mutually agreeing on an intensity score and attribute characterization for the particular sensory attribute(s) being assayed. A sensory panel using a roundtable consensus approach may include 2, 3, 4, 5, 6, or more panelists. Consensus intensity scales can range from 0 to 6 (i.e., 0=not detected, 1=trace, 2=slight, 3=moderate, 4=definite, 5=strong, 6=extreme) or 0 to 9 (i.e., 0=not detected, 1=trace, 2=faint, 3=slight, 4=mild, 5=moderate, 6=definite, 7=strong, 8=very strong, 9=extreme). For a given set of samples, the panelists will identify and agree on a lexicon of sensory attribute, including, if applicable, reference or standardized samples (also referred to as sensory anchors) for a particular sensory attribute. The reference sample(s) used for a given sensory attribute(s) will depend on the samples being assayed and the lexicon of sensory attributes determined by the panel. One of skill in the art will recognize the appropriate lexicon and reference or standard samples necessary for sensory assessment of a given sample(s).

In some aspects, the samples are scored and evaluated by panelists independently after panelists have agreed upon or been instructed in a lexicon of sensory attributes and intensity scores including, if applicable, assay specific calibration on reference samples (also referred to as sensory anchors) for a particular sensory attribute. Examples of common reference samples are described below. Panelists may evaluate samples in replicate and may be blinded to the samples they are testing. Samples being tested may be provided to the panelists randomly or in a sequential order. In some aspects, samples may be tested by panelists using a randomized balanced sequential order. Scores from individual panelists are then assessed using standard statistical analysis methods to determine an average sensory intensity score. One of skill in the art will recognize the appropriate lexicon and reference or standard samples necessary for sensory assessment of a given sample(s) as well as the appropriate statistical analysis methods.

As used herein, “randomized balanced sequential order” refers to the order in which samples are presented in which the order is randomized but across all panelists all possible orders of the samples will be presented to remove bias for the samples being tested in a particular order. For example, for a randomized balanced sequential order of two samples, there would be an equal likelihood that a given panelist receives sample 1 before sample 2 and sample 2 before sample 1. In an example with three samples (i.e., samples 1, 2, and 3), a randomized balanced sequential order would include an equal likelihood that panelists receiving samples in the following orders: (i) 1, 2, 3; (ii) 1, 3, 2; (iii) 2, 1, 3; (iv) 2, 3, 1; (v) 3, 2, 1; (vi) 3, 1, 2.

A sensory attribute(s) of a given composition may be evaluated in comparison to one or more reference or anchor samples. For example, sodium chloride solutions can be used by experienced panelists as saltiness anchors to assess the relative intensity of saltiness for a given composition; sucrose solutions can be used by experienced panelists as sweetness anchors to assess the relative intensity of sweetness for a given composition; citric acid solutions can be used by experienced panelists as sourness anchors to assess the relative intensity of sourness for a given composition; caffeine solutions can be used by experienced panelists as bitterness anchors to assess the relative intensity of bitterness for a given composition; and monosodium glutamate (MSG) solutions can be used by experienced panelists as umami anchors to assess the relative intensity of umami for a given composition. Experienced panelists can be presented with a solution to assess sensory attributes, e.g., 10-20 mL of a sample. Panelists will dispense approximately 3-4 mL of each solution into their own mouths, disperse the solution by moving their tongues, and record a value for the particular sensory attribute being tested. If multiple solutions are to be tested in a session, the panelists may cleanse their palates with water between samples. For example, a roundtable assessment of saltiness, sweetness, sourness, umami, and the like can assign a scale of 0 to 9 with, e.g., a score of 0 indicating no saltiness and a score of 9 indicating extreme saltiness (0=not detected, 1=trace, 2=faint, 3=slight, 4=mild, 5=moderate, 6=definite, 7=strong, 8=very strong, 9=extreme). Equivalent scales and methodologies can be used for sweet, bitter, sour, and umami sensory attributes.

As a further example, saltiness of a composition can be tested by a panel of at least two panelists. The panelists can use a standard range of 0.18% (wt), 0.2% (wt), 0.35% (wt), 0.5% (wt), 0.567% (wt), 0.6% (wt), 0.65% (wt), and 0.7% (wt) sodium chloride solutions in water corresponding to a saltiness intensity value of 2, 2.5, 5, 8.5, 10, 11, 13, and 15, respectively. A skilled artisan will recognize that depending on the sample/composition being tested, the number and range of standard solutions may be changed (e.g., using only the solutions corresponding to the 2, 2.5, and 5 saltiness intensity values). For each test composition, the panelists dispense approximately 2-5 mL, for liquid compositions or solutions prepared with water, or 5-10 g, for solid compositions, of each composition into their own mouths, disperses the composition by moving their tongues/chewing, and records a saltiness intensity value between 0 and 15 for each composition based on comparison to the aforementioned standard sodium chloride solutions. Between tasting compositions, the panelists are able to cleanse their palates with water. The panelists also can taste the standard 0.18%, 0.2%, 0.35%, 0.5%, 0.567%, 0.6%, 0.65%, and 0.7% sodium chloride solutions ad libitum between tasting test solutions to ensure recorded saltiness intensity values are accurate against the scale of the standard sodium chloride solutions. The temperature at which the test is conducted may be specific to the sample beginning tested, e.g., samples may be tested at 22 °C (e.g., room temperature), at 0 °C (e.g., for frozen samples), or between 60-80 °C (e.g., a cooked sample served warm). One skilled in the art will recognize the appropriate temperature for testing a given sample. This test is referred to herein as the “Standardized Saltiness Intensity Test.”

Sourness of a composition can be tested by a panel of at least two panelists. The panelists can use a standard range of 0.035% (wt), 0.05% (wt), 0.07% (wt), 0.15% (wt), and 0.2% (wt) citric acid solutions in water corresponding to a sourness intensity value of 2, 3, 5, 10, and 15, respectively. A skilled artisan will recognize that depending on the sample/composition being tested, the number and range of standard solutions may be changed (e.g., using only the solutions corresponding to the 2 and 7 sourness intensity values). For each test composition, the panelists dispense approximately 2-5 mL, for liquid compositions or solutions prepared with water, or 5-10 g, for solid compositions, of each composition into their own mouths, disperses the composition by moving their tongues/chewing, and records a sourness intensity value between 0 and 15 for each composition based on comparison to the aforementioned standard citric acid solutions. Between tasting compositions, the panelists are able to cleanse their palates with water. The panelists also can taste the standard 0.035%, 0.05%, 0.07%, 0.15%, and 0.2% citric acid solutions ad libitum between tasting test solutions to ensure recorded sourness intensity values are accurate against the scale of the standard citric acid solutions. The temperature at which the test is conducted may be specific to the sample beginning tested, e.g., samples may be tested at 22 °C (e.g., room temperature), at 0 °C (e.g., for frozen samples), or between 60-80 °C (e.g., a cooked sample served warm). One skilled in the art will recognize the appropriate temperature for testing a given sample. This test is referred to herein as the “Standardized Sourness Intensity Test.”

Bitterness of a composition can be tested by a panel of at least two panelists. The panelists can use a standard range of 0.0125% (wt), 0.01875% (wt), 0.025% (wt), 0.031% (wt), 0.07% (wt), and 0.12% (wt) caffeine solutions in water corresponding to a bitterness intensity value of 2, 3, 4, 5, 10, and 15, respectively. A skilled artisan will recognize that depending on the sample/composition being tested, the number and range of standard solutions may be changed (e.g., using only the solutions corresponding to the 2, 3, and 5 bitterness intensity values). For each test composition, the panelists dispense approximately 2-5 mL, for liquid compositions or solutions prepared with water, or 5-10 g, for solid compositions, of each composition into their own mouths, disperses the composition by moving their tongues/chewing, and records a bitterness intensity value between 0 and 15 for each composition based on comparison to the aforementioned standard caffeine solutions. Between tasting compositions, the panelists are able to cleanse their palates with water. The panelists also can taste the standard 0.0125%, 0.01875%, 0.025%, 0.031%, 0.07%, and 0.12% caffeine solutions ad libitum between tasting test solutions to ensure recorded bitterness intensity values are accurate against the scale of the standard caffeine solutions. The temperature at which the test is conducted may be specific to the sample beginning tested, e.g., samples may be tested at 22 °C (e.g., room temperature), at 0 °C (e.g., for frozen samples), or between 60-80 °C (e.g., a cooked sample served warm). One skilled in the art will recognize the appropriate temperature for testing a given sample. This test is referred to herein as the “Standardized Bitterness Intensity Test.”

Sweetness of a composition can be tested by a panel of at least two panelists. The panelists can use a standard range of 2% (wt), 5% (wt), 8% (wt), 10% (wt), and 15% (wt) sucrose solutions corresponding to a sweetness intensity value of 2, 5, 8, 10, and 15, respectively. A skilled artisan will recognize that depending on the sample/composition being tested, the number and range of standard solutions may be changed (e.g., using only the solutions corresponding to the 2, 5, and 8 sweetness intensity values). For each test composition, the panelists dispense approximately 2-5 mL, for liquid compositions or solutions prepared with water, or 5-10 g, for solid compositions, of each composition into their own mouths, disperses the composition by moving their tongues/chewing, and records a sweetness intensity value between 0 and 15 for each composition based on comparison to the aforementioned standard sucrose solutions. Between tasting compositions, the panelists are able to cleanse their palates with water. The panelists also can taste the standard 2%, 5%, 8%, 10%, and 15% sucrose solutions ad libitum between tasting test solutions to ensure recorded sweetness intensity values are accurate against the scale of the standard sucrose solutions. The temperature at which the test is conducted may be specific to the sample beginning tested, e.g., samples may be tested at 22 °C (e.g., room temperature), at 0 °C (e.g., for frozen samples), or between 60-80 °C (e.g., a cooked sample served warm). One skilled in the art will recognize the appropriate temperature for testing a given sample. This test is referred to herein as the “Standardized Sweetness Intensity Test.”

Umami of a composition can be tested by a panel of at least two panelists. The panelists can use a standard range of 0.75% (wt) and 0.125% (wt) monosodium glutamate (MSG) solutions corresponding to an umami intensity value of 4 and 6.5, respectively. A skilled artisan will recognize that depending on the sample/composition being tested, the number and range of standard solutions may be changed (e.g., adding additional umami solutions if the umami intensity is expected to be appreciably outside of the umami intensity value of 4-6.5). For each test composition, the panelists dispense approximately 2-5 mL, for liquid compositions or solutions prepared with water, or 5-10 g, for solid compositions, of each composition into their own mouths, disperses the composition by moving their tongues/chewing, and records an umami intensity value between 0 and 15 for each composition based on comparison to the aforementioned standard MSG solutions. Between tasting compositions, the panelists are able to cleanse their palates with water. The panelists also can taste the standard 0.075% and 0.125% MSG solutions ad libitum between tasting test solutions to ensure recorded umami intensity values are accurate against the scale of the standard MSG solutions. The temperature at which the test is conducted may be specific to the sample beginning tested, e.g., samples may be tested at 22 °C (e.g., room temperature), at 0 °C (e.g., for frozen samples), or between 60-80 °C (e.g., a cooked sample served warm). One skilled in the art will recognize the appropriate temperature for testing a given sample. This test is referred to herein as the “Standardized Umami Intensity Test.”

A control sample is typically used as a reference point or for comparison purposes. The control sample can be a composition such as a composition as described herein, but that has not been fermented or contacted by the L. citreum bacterium. Similarly, the control sample may be a reference sample with similar composition of protein, sweetness, etc. but made with different ingredients, such as the pea protein fermentate described herein. Other than the pea protein fermentate, the control sample is otherwise the same, and it should contain the same component(s) and other ingredients at the same relative concentrations. Other standard samples are commonly used in sensory panels, for example standard samples used to evaluate intensity of sensory attributes as outlined above.

This disclosure is not limited to sensory testing by experienced or trained panelists. For example, it is possible to utilize untrained and inexperienced panelists. However, in the case of untrained and inexperienced panelists, a greater number of these panelists is usually necessary to provide reproducible results, which will typically focus on subjective attributes such as preference or overall liking. Similarly, untrained, and inexperienced panelists may be asked to evaluate relative changes in a given sensory attribute between two samples. For example, if a particular sample is more or less salty, more or less sweet, more or less bitter, etc., than a reference sample.

An exemplified sensory assay and test criteria for further sensory attributes are described in the Examples provided in this disclosure.

### Dessert Compositions

The pea protein fermentates described herein may be used in the preparation of a dessert composition. The dessert composition may be a yogurt, a pudding, a custard, or a similar creamy spoonable dessert. The dessert composition may be a vegan dessert composition. As used herein, “vegan” refers to compositions free of any animal, or animal derived, products or ingredients.

In general, the dessert compositions include the pea protein fermentate described herein. The dessert compositions may additionally include one or more additional ingredients, including but not limited to, a sweetener, a flavor, a seasoning, a lipid composition, water, fiber, starch, a hydrocolloid, lecithin, a preservative, an acid, and combinations thereof.

The dessert composition described herein may include one or more lipid compositions, for example a fat, an oil, or combinations thereof. In general, fats refer to lipid compositions that are solid at room temperature, whereas oils are liquid at room temperature. The lipid compositions may include saturated fatty acids (also referred to as “saturated fats”), unsaturated fatty acids (also referred to as “unsaturated fats”), or combinations thereof. The lipid composition may include, but are not limited to, vegetable oil, coconut oil, palm oil, sunflower oil, soy oil, canola oil, or combinations thereof. The dessert composition may optionally include between 1% and 80%, between 1% and 70%, between 1% and 10%, between 1% and 5%, between 5% and 30%, between 10% and 25%, between 10% and 75%, or between 15% and 70% by weight of a lipid composition depending on the type of dessert. An ordinarily skilled artisan will understand the appropriate lipid composition inclusion rate for a given dessert composition.

The dessert composition may include water. For example, the dessert composition may optionally include between 1% and 80%, between 5% and 75%, between 15% and 70%, between 45% and 65%, between 50% and 60%, between 1% and 20%, or between 5% and 15% by weight of water depending on the type of dessert composition. One of skill in the art will recognize the appropriate amount of water, if any, to include in a given dessert composition.

The dessert composition may include fiber. The fiber may include, but is not limited to, pectin, apple fiber, psyllium, flax fiber, rice bran extract, Konjac flour, and the like. The dessert composition may include between 0.01% (wt) and 3% (wt), between 0.05% (wt) and 2% (wt), or between 0.1% (wt) and 2% (wt) of fiber. The dessert composition may include fiber in an amount up to 0.5% (wt), up to 1% (wt), up to 1.5% (wt), up to 2% (wt), up to 2.5% (wt), or up to 3% (wt).

The dessert composition may include starch. The starch may include a pregelatinized starch, a modified starch, or combinations thereof. The starch may include, but is not limited to, corn starch, potato starch, tapioca starch, and the like. The dessert composition may include between 0.5% (wt) and 25% (wt), between 1.0% (wt) and 20% (wt), or between 2% (wt) and 18% (wt) of starch. Alternatively, the dessert composition may be free of starch. Without wishing to be bound by any particular theory, method, or mode of action, it is believed that inclusion of the pea protein fermentate described herein in a dessert composition will reduce or eliminate the need to include a starch while maintaining the same or similar physical properties. Accordingly, the dessert composition may include at least 50% less, at least 60% less, at least 70%, less at least 80% less, or at least 90% less starch than an equivalent dessert composition lacking the pea protein fermentate by having the same viscosity and/or other physical properties.

The dessert composition may include a hydrocolloid. For example, the dessert composition may include guar gum, xanthan gum, locust bean gum, carrageenan, cellulose, konjac gum, and combinations thereof. The dessert composition may include between 0.01% and 5%, between 0.05% and 4.5%, between 0.1% and 4.0%, or between 0.5% and 3.8% by weight of hydrocolloid. The dessert composition may include up to 5%, up to 4.5%, up to 4.0%, up to 3.8%, up to 3.5%, up to 2.5%, up to 2.0%, or up to 1.0% by weight of hydrocolloid. Alternatively, the dessert composition may be free of hydrocolloids. Without wishing to be bound by any particular theory, method, or mode of action, it is believed that inclusion of the pea protein fermentate described herein in a dessert composition will reduce or eliminate the need to include a hydrocolloid while maintaining the same or similar physical properties. Accordingly, the dessert composition may include at least 50% less, at least 60% less, at least 70%, less at least 80% less, or at least 90% less hydrocolloid than an equivalent dessert composition lacking the pea protein fermentate by having the same viscosity and/or other physical properties.

The dessert composition may include lecithin. The dessert composition may include between 0.010% and 10%, between 0.05% and 8.0%, or between 0.10% and 5% by weight lecithin.

The dessert composition may include a preservative. For example, the dessert composition may include a preservative such as, but not limited to potassium sorbate. The dessert composition may include a preservative in an amount up to 0.1%, up to 0.5%, or up to 1.0% by weight of the dessert composition.

The dessert composition may include a flavor or seasoning. For example, the dessert composition may include a natural or artificial flavor(s) and/or seasonings. Seasonings and/or flavors may include, but are not limited to, sweetener(s), salt (e.g., sodium chloride, potassium chloride, and the like), cocoa, chocolate, cinnamon, vanilla, nutmeg, coconut, almond, combinations thereof, and the like. The dessert composition may include between 1% and 20%, between 1.5% and 10%, between 5% and 20%, or between 2% and 18% of sweetener. The dessert composition may be free of any sweetener. The dessert composition may include between 0.001% and 3.0%, between 0.01% and 2.0%, or between 0.025% and 1.75% of a salt. The dessert composition may be free of salt.

The dessert composition may include a sweetener. Suitable sweeteners are known and described in the art. The sweetener can be at least one of a non-caloric sweetener or a caloric sweetener. The sweetener can be any type of sweetener, for example, a sweetener obtained from a plant or plant product, or a physically or chemically modified sweetener obtained from a plant, or a synthetic sweetener. Exemplary sweeteners include steviol glycosides, mogrosides, sucrose, fructose, glucose, erythritol, maltitol, lactitol, sorbitol, mannitol, xylitol, tagatose, trehalose, galactose, rhamnose, cyclodextrin (e.g., a-cyclodextrin, β-cyclodextrin, and γ-cyclodextrin), ribulose, threose, arabinose, xylose, lyxose, allose, altrose, mannose, idose, lactose, maltose, invert sugar, isotrehalose, neotrehalose, palatinose or isomaltulose, erythrose, deoxyribose, gulose, idose, talose, erythrulose, xylulose, psicose, turanose, cellobiose, glucosamine, mannosamine, fucose, fuculose, glucuronic acid, gluconic acid, glucono-lactone, abequose, galactosamine, xylo-oligosaccharides (xylotriose, xylobiose and the like), gentio-oligoscaccharides (gentiobiose, gentiotriose, gentiotetraose and the like), galacto-oligosaccharides, sorbose, ketotriose (dehydroxyacetone), aldotriose (glyceraldehyde), nigero-oligosaccharides, fructooligosaccharides (kestose, nystose and the like), maltotetraose, maltotriol, tetrasaccharides, mannan-oligosaccharides, malto-oligosaccharides (maltotriose, maltotetraose, maltopentaose, maltohexaose, maltoheptaose and the like), dextrins, lactulose, melibiose, raffinose, rhamnose, ribose, sucralose, acesulfame K, aspartame, saccharin, coupling sugars, soybean oligosaccharides, and combinations thereof. D- or L-configurations can be used when applicable.

The dessert composition may include an acid. Suitable acids include, but are not limited to, citric acid, lactic acid, sorbic acid, malic acid, combinations thereof, and the like. The dessert composition may include an acid in an amount up to 0.001%, up to 0.005%, up to 0.01%, up to 0.1%, up to 1.0%, up to 1.5%, or up to 2.0% of the dessert composition. The dessert composition may include between 0.0001% and 2.0%, between 0.0002% and 1.5%, between 0.0003% and 1.0% by weight of an acid.

### EXAMPLES

The invention is further described in detail by reference to the following experimental examples. These examples are provided for purposes of illustration only and are not intended to be limiting unless otherwise specified. Thus, the invention should in no way be construed as being limited to the following examples, but rather should be construed to encompass any and all variations which become evident as a result of the teaching provided herein.

### Example 1—Pea Protein Fermentation with Leuconostoc sp.

Three strains of Leuconostoc sp., isolated from different ecological systems, were used to ferment a pea protein isolate. Each of the three strains, B3K7, C22B11, and C18X24, were identified and allocated to the species Leuconostoc citreum (B3K7 and C22B11) or Leuconostoc pseudomesenteroides (C18X24) using MALDI-TOF_MS fingerprinting. The obtained MALDI profiles were identical and the profile from B3K7 was used as a representative of the cluster. The B3K7 strain underwent 16s rRNA gene sequencing and whole genome sequencing to confirm the species allocation.

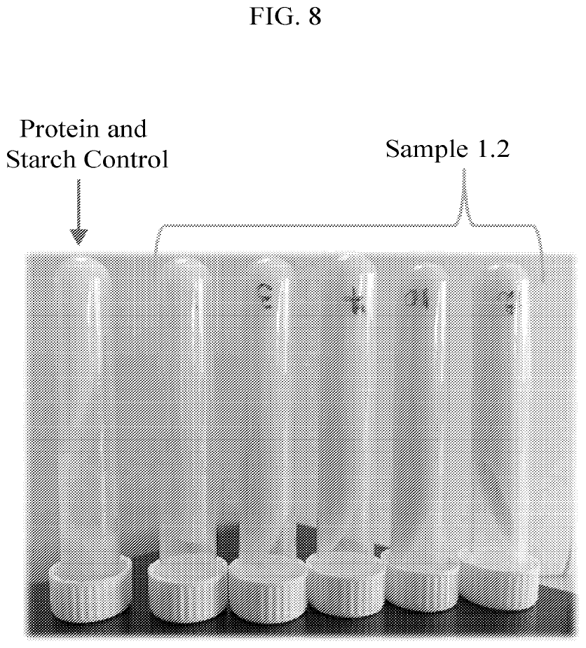

Table 1 outlines combinations of pea protein cultures and Leuconostoc citreum or Leuconostoc pseudomesenteroides strains used for pea protein fermentations. Each fermentate was inoculated with 3.8 wt % of the indicated strain of Leuconostoc sp. Fermentations were carried out in water. The cultures outlined in Table 1 were fermented at 25 °C for 24 hours without stirring. The resulting samples were then evaluated for syneresis, aspect, taste, and texture. Syneresis was evaluated by centrifuging the fermentate sample at 5,000 g for 10 minutes. A comparison of the syneresis observed in a control starch and protein suspension to the lack of syneresis observed for sample 1.2 is shown in FIG. 8. Aspect was evaluated visually.

Assays were carried out to characterize the sensory attributes of the samples. Sensory attributes (taste and texture) were evaluated by a panel of 12 individuals that are experienced in plant-protein sensory testing. The experienced panelists assessed sensory attributes such as, but not limited to, green pea flavor, sourness, chalky, nutty flavor, sweetness, and texture. To test each sample, the experienced panelists dispensed a portion of each sample into their own mouths, dispersed the sample around their mouth, and recorded their observations. Texture and aspect observations were also assessed visually.

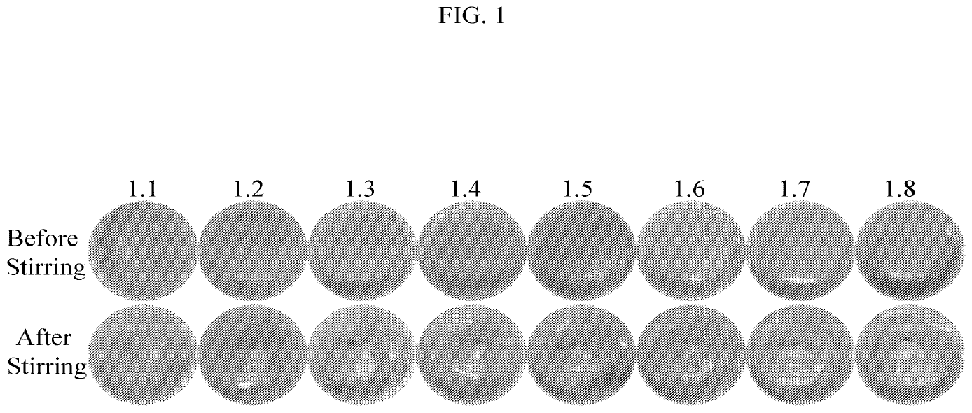

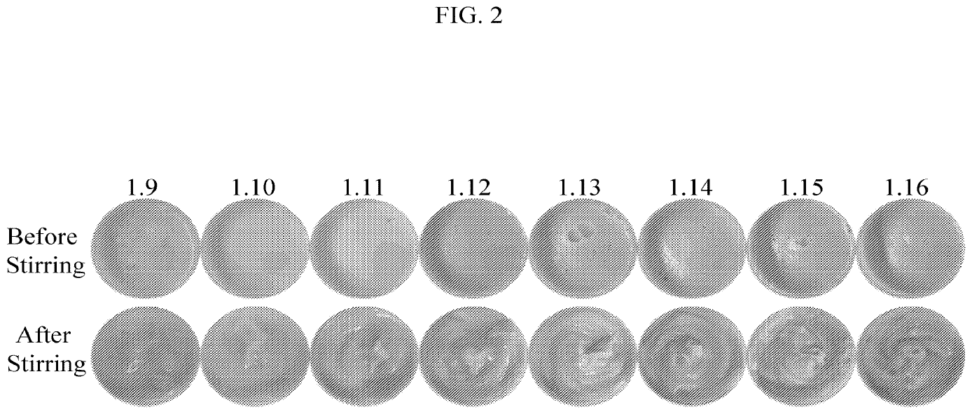

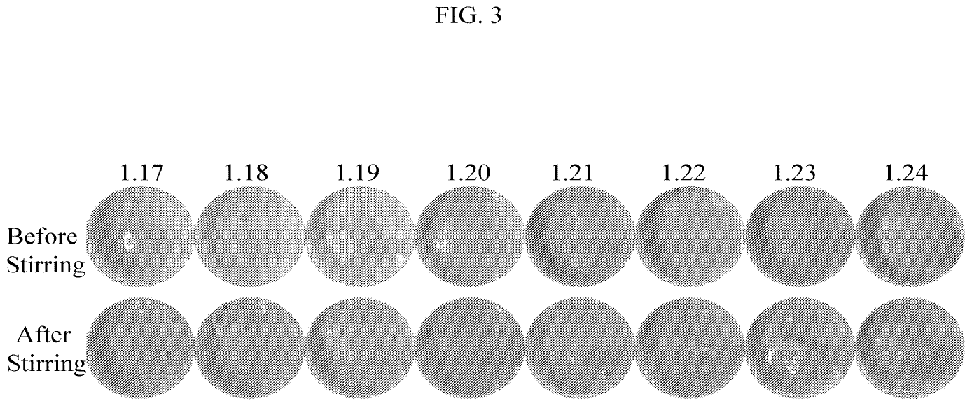

Photos of the samples outlined in Table 1, both before and after manually stirring for approximately 2-5 seconds, are provided in FIGS. 1, 2, and 3.

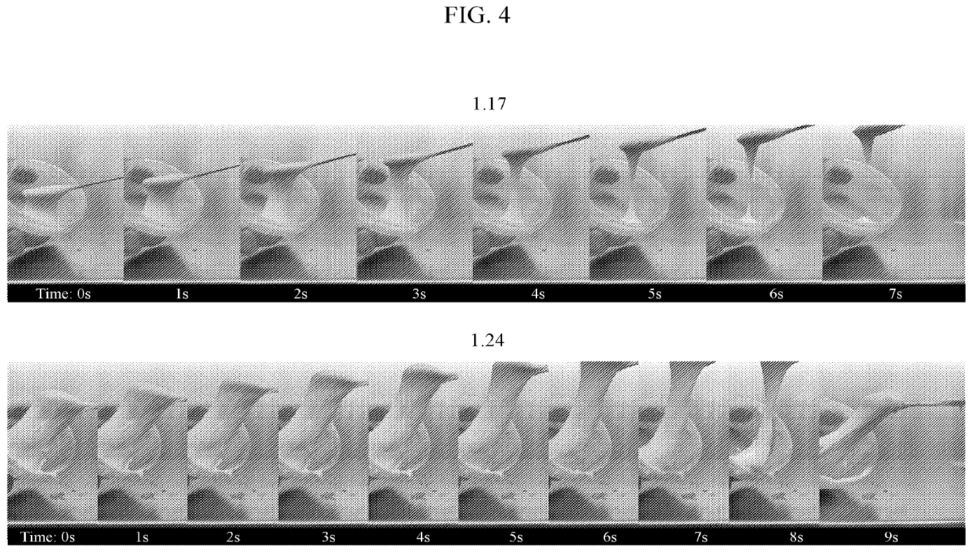

Overall, while there is some strain-to-strain variability in the fermentate samples, all strains demonstrated significant viscosity effects, acidification of the sample, and flavor modification reducing the grassy and green pea flavor of the samples. Fermentates produced using Leuconostoc citreum B3K7 were sourer with a very smooth glossy and shiny texture, rich in acidic and umami notes. Leuconostoc citreum C22B11 began to thicken faster (approximately 4-5 hours) and produced less sour but more nutty notes and the texture appeared thicker with very stiff peaks and manual stirring. Leuconostoc pseudomesenteroides C18X24 fermentates produced samples with very ropy and slimy texture, similar to a melted cheese (see FIG. 4).

TABLE 1

| | | Pea Protein | | | | | |
|---|---|---|---|---|---|---|---|
| | | Isolate | Sucrose | | | | |
| Sample | Strain | Concentration | Concentration | Syneresis | Aspect | Taste | Texture |
| 1.1 | B3K7 | 4% | 10% | yes | glossy, shiny, gel-like | strong sour | rich, thick |
| 1.2 | | 4% | 15% | no | glossy, shiny, gel-like | sour, sweet | rich, thick |
| 1.3 | | 6% | 10% | no | glossy, shiny, gel-like | neutral, little sour, pleasant | rich, thick |
| 1.4 | | 6% | 15% | no | glossy, shiny, gel-like | neutral, little sour, | rich, very thick |
| | | | | | | little sweet, pleasant | |
| 1.5 | | 8% | 10% | no | glossy, shiny, gel-like | neutral, little sour, slightly pea | rich, very thick |
| 1.6 | | 8% | 15% | no | glossy, shiny, gel-like | neutral, little sour, pleasant, | rich, very thick, stiff peaks, |
| | | | | | | slightly chalky | paste |
| 1.7 | | 10% | 10% | no | glossy, shiny, gel-like | neutral, little sour, slightly pea, | rich, very thick, very stiff |
| | | | | | | chalky | peaks, paste |
| 1.8 | | 10% | 15% | no | glossy, shiny, gel-like | neutral, little sour, | rich, very thick, very stiff |
| | | | | | | sweet, slightly pea | peaks, paste |
| 1.9 | C22B11 | 4% | 10% | yes | glossy, shiny, gel-like | slightly sour, nutty | rich, thick |
| 1.10 | | 4% | 15% | no | glossy, shiny, gel-like | slightly sour, sweet, nutty | rich, thick |
| 1.11 | | 6% | 10% | no | glossy, shiny, gel-like | neutral, nutty | rich, thick |
| 1.12 | | 6% | 15% | no | glossy, shiny, gel-like | neutral, little sweet, | rich, very thick |
| | | | | | | slightly sour, pleasant | |
| 1.13 | | 8% | 10% | no | glossy, shiny, gel-like | neutral, nutty | rich, very thick |
| 1.14 | | 8% | 15% | no | glossy, shiny, gel-like | neutral, little sour, pleasant, | rich, very thick, stiff peaks, |
| | | | | | | slightly chalky | paste |
| 1.15 | | 10% | 10% | no | glossy, shiny, gel-like | neutral, little sour, slightly | rich, very thick, very stiff |
| | | | | | | chalky, nutty | peaks, paste |
| 1.16 | | 10% | 15% | no | glossy, shiny, gel-like | neutral, little sour, | rich, very thick, very stiff |
| | | | | | | sweet, nutty | peaks, paste |
| 1.17 | C18X24 | 4% | 10% | yes | glossy, shiny, gooey | strong sour | sticky, ropy, slimy |
| 1.18 | | 4% | 15% | no | glossy, shiny, gooey | strong sour | sticky, ropy, slimy |
| 1.19 | | 6% | 10% | no | glossy, shiny, gooey | strong sour | sticky, ropy, slimy |
| 1.20 | | 6% | 15% | no | glossy, shiny, gooey | strong sour | sticky, ropy, slimy |
| 1.21 | | 8% | 10% | no | glossy, shiny, gooey | sour, chalky | sticky, ropy, slimy |
| 1.22 | | 8% | 15% | no | glossy, shiny, gooey | sour, chalky | sticky, ropy, slimy |
| 1.23 | | 10% | 10% | no | glossy, shiny, gooey | sour, chalky | sticky, ropy, slimy |
| 1.24 | | 10% | 15% | no | glossy, shiny, gooey | sour, chalky | sticky, ropy, slimy |

### Example 2—Viscosity and Acidification

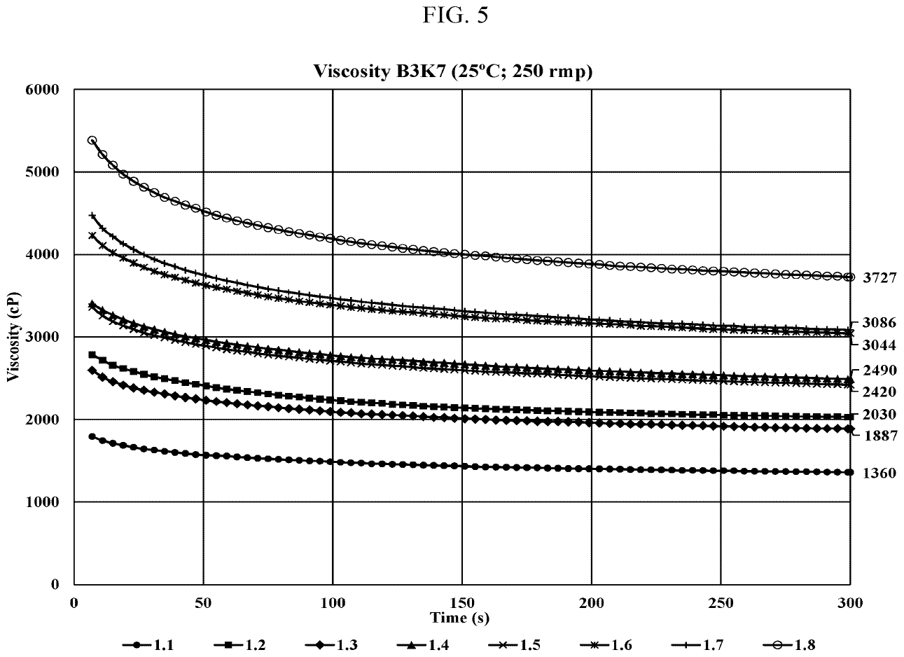

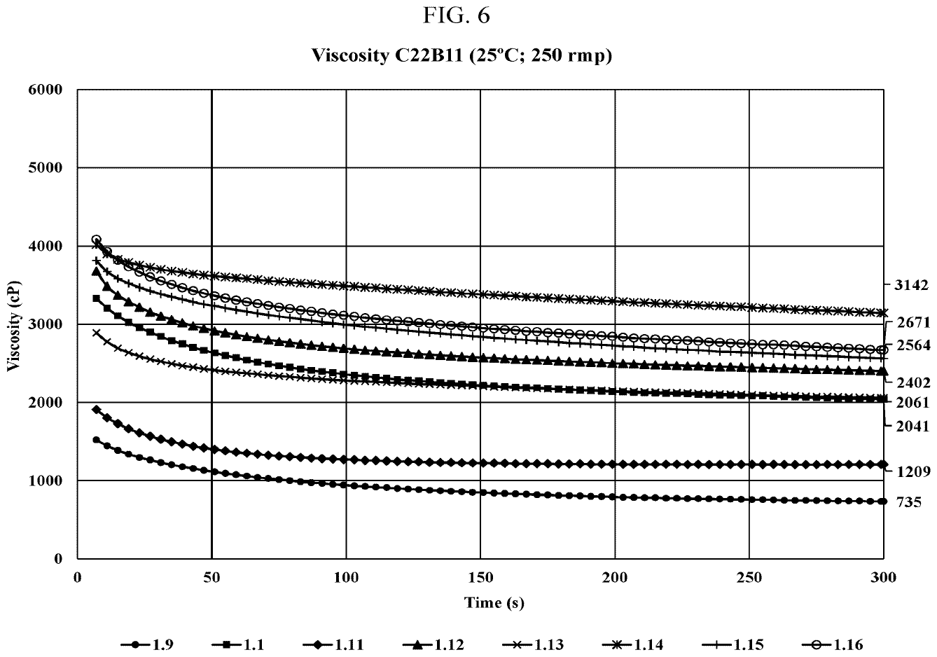

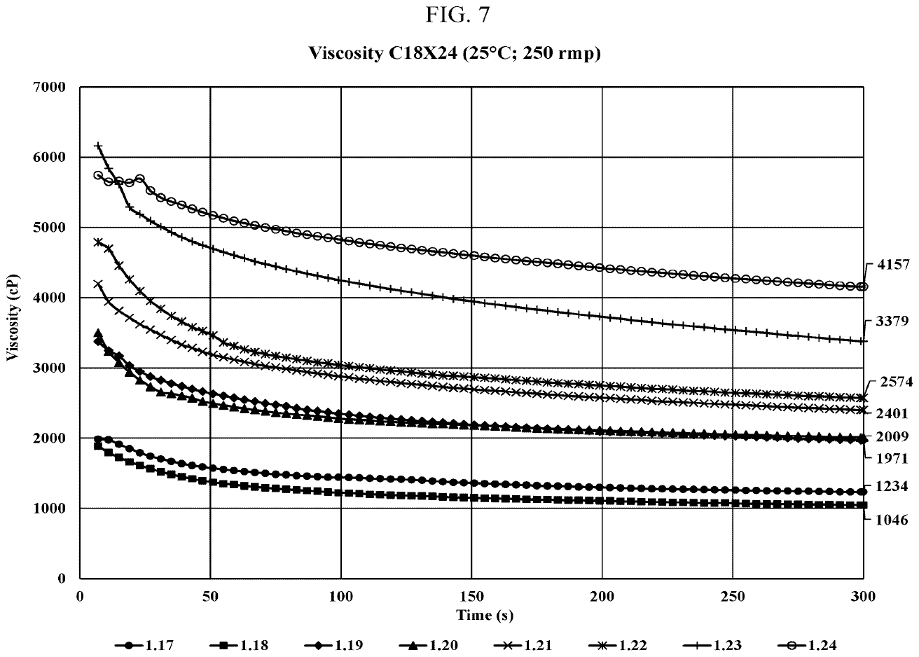

Viscosity of the samples outlined in Table 1 was tested alongside a blank pea protein sample containing pea protein isolate, sucrose, and water but lacking inoculation with any Leuconostoc sp. strain. Viscosity (cP) was measured at 25 °C and 250 rpm using a Rapid Visco Analyzer (RVA). As demonstrated in FIGS. 5-7 and Table 2, all samples showed significantly increased viscosity relative to the non-fermented pea protein blank. However, there is some strain variability in the absolute increase in viscosity over the blank.

Samples were also subjected to two consecutive freeze-thaw cycles and viscosity was measured following the freeze-thaw and found to be consistent with the samples prior to freezing. Visual analysis also showed no changes or loss of thickness upon freezing and thawing.

The pH level of each fermentate varied based on strain, protein concentration and sucrose concentration. Overall, pH increased (acidification decreased) as the protein concentration increased. This effect was most apparent for the B3K7 strain. For strains C22B11 and C18X24, while there was an increase in pH with increasing protein concentration, the pH of samples produced with greater than 6% protein was less significant.

TABLE 2

| | | Viscosity | |
|---|---|---|---|
| Strain | Sample | (cP) | pH |
| none | blank | 49 | 7.4 |
| B3K7 | 1.1 | 1360 | 4.23 |
| | 1.2 | 2030 | 4.22 |
| | 1.3 | 1887 | 4.34 |
| | 1.4 | 2490 | 4.36 |
| | 1.5 | 2420 | 4.52 |
| | 1.6 | 3044 | 4.47 |
| | 1.7 | 3086 | 4.66 |
| | 1.8 | 3727 | 4.68 |
| C22B11 | 1.9 | 735 | 4.24 |
| | 1.10 | 2041 | 4.19 |
| | 1.11 | 1209 | 4.25 |
| | 1.12 | 2402 | 4.24 |
| | 1.13 | 2061 | 4.38 |
| | 1.14 | 3142 | 4.39 |
| | 1.15 | 2564 | 4.47 |
| | 1.16 | 2671 | 4.48 |
| C18X24 | 1.17 | 1234 | 4.24 |
| | 1.18 | 1046 | 4.22 |
| | 1.19 | 1971 | 4.27 |
| | 1.20 | 2009 | 4.41 |
| | 1.21 | 2401 | 4.36 |
| | 1.22 | 2574 | 4.43 |
| | 1.23 | 3379 | 4.44 |
| | 1.24 | 4157 | 4.44 |

### Example 3—Compositional Analysis

Sample 1.2 was chosen for further compositional analysis based on the aspect, spoonability, and taste. Sample 1.2 was analyzed by UPLC-RI to quantify simple carbohydrates and HPLC-RI to quantify sugar alcohols and organic acids. The sucrose (15%) present at the beginning of fermentation was completely utilized by the end of fermentation and fructose, alpha-glucan, and mannitol were produced. Lactic acid and acetic acid were also present in the resulting fermentate. While the alpha-glucan concentration was not independently quantified, the value was obtained by subtracting other identified carbohydrates from the total carbohydrates present. The compositional analysis of sample 1.2 is reported in Table 3.

TABLE 3

| Component % | Sample 1.2 |
|---|---|
| Protein | 3.3 |
| Carbohydrates | 16.1 |
| Glucose | 0.0 |
| Fructose | 6.0 |
| Sucrose | 0.0 |
| Alpha-glucan | 6.5 |
| Polyols | 2.0 |
| Dietary fiber | 0.2 |
| Organic acids | 1.4 |
| Fat | 0.3 |
| Ash | 0.2 |
| Water | 79.1 |
| Total Solids | 19 |

### Example 4—Pea Protein Dessert

Aspect and consistency of the pea protein fermentate compositions of samples 1.2, 1.10, and 1.18 were compared to a reference vegan dessert formulation, outlined in Table 4. Compositional analysis of the reference vegan dessert formulation is provided in Table 5. For comparison the reference vegan dessert was formulated to match the protein content and sweetness of the pea protein fermentate of sample 1.2. The reference vegan dessert formulation was prepared by mixing the dry ingredients (pea protein, sucrose, starch, and carrageenan) and adding the mixed dry ingredients to the water at room temperature (about 25 °C) with stirring at 800 rpm. After the mixed dry ingredients were hydrated, the entire mixture was pasteurized by heating to 95 °C. The pasteurized mixture was cooled to between 50-70 °C, then refrigerated (4 °C) overnight. Samples were assayed the following day for sensory attributes and aspect.

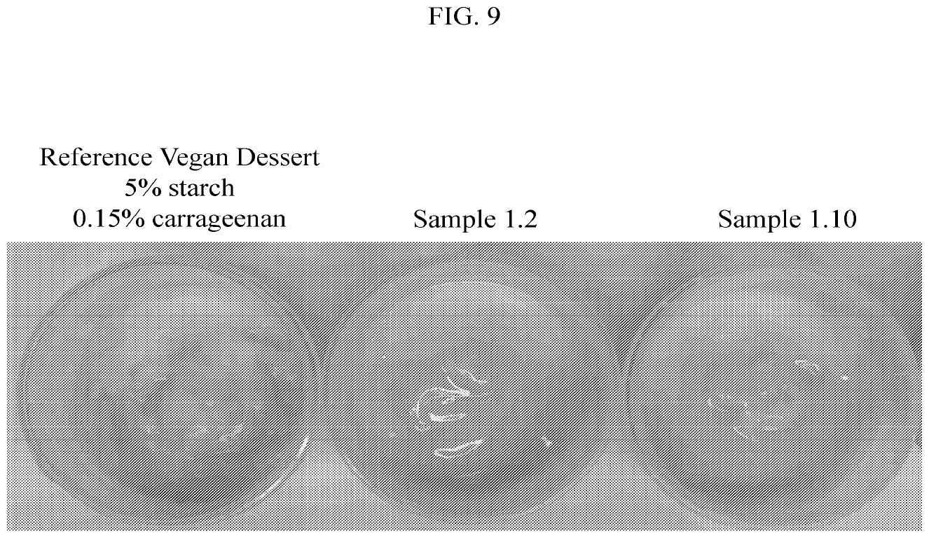

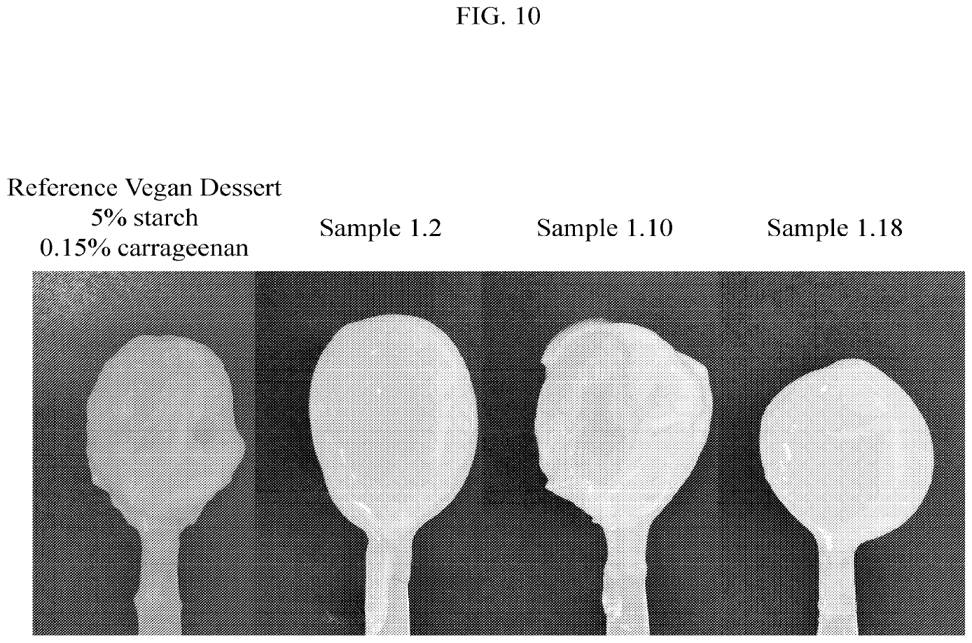

A visual comparison of the aspect of the reference vegan dessert and the vegan desserts prepared with samples 1.2, 1.10, and 1.18 is provided in FIGS. 9 and 10. Overall, the vegan desserts prepared with samples 1.2 and 1.10 had a smoother texture and glossier and shinier appearance than the reference vegan dessert sample.

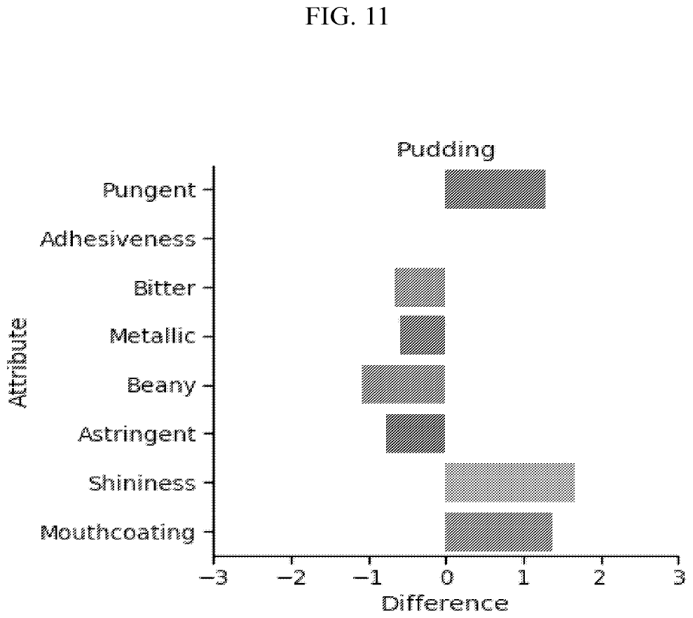

Additionally, sensory attributes of both the reference vegan dessert and sample 1.2 were compared. Relative to the reference vegan dessert, sample 1.2 was perceived as more pungent, less bitter, less metallic, less beany, less astringent, shinier, and to have a better mouthcoating than the reference vegan dessert when sampled by a panel of 4 individuals experienced in sensory evaluation. See FIG. 11.

TABLE 4

| Ingredient | wt % |
|---|---|
| Pea protein isolate | 4.0 |
| Sucrose | 7.0 |
| Hydroxypropylated starch | 5.0 |
| Carrageenan | 0.015 |
| Water | 84.0 |

TABLE 5

| | Reference Vegan |
|---|---|
| Component % | Dessert |
| Protein | 3.3 |
| Carbohydrates | 12.5 |
| Sucrose | 7.0 |
| Dietary fiber | 0.2 |
| Fat | 0.3 |
| Ash | 0.2 |
| Water | 84 |
| Total Solids | 16 |

### Example 5—Comparative Pea Protein Fermentation

Pea protein fermentation by three strains of Leuconostoc citreum and one strain of Leuconostoc pseudomesenteroides were compared to fermentation by the dairy-isolated lactic acid bacteria culture containing Streptococcus thermophilus and Lactobacillus bulgaricus known under the tradename “YO-MIX 433.” This bacterial culture is known in the art as a yogurt culture and is sold by DANISCO®. Pea protein fermentation using YO-MIX 433 was previously described in US 2020/296982.

In this Example, pea protein fermentation was done using four different strains/cultures and three different fermentation protocols, as outlined in Tables 6 and 7.

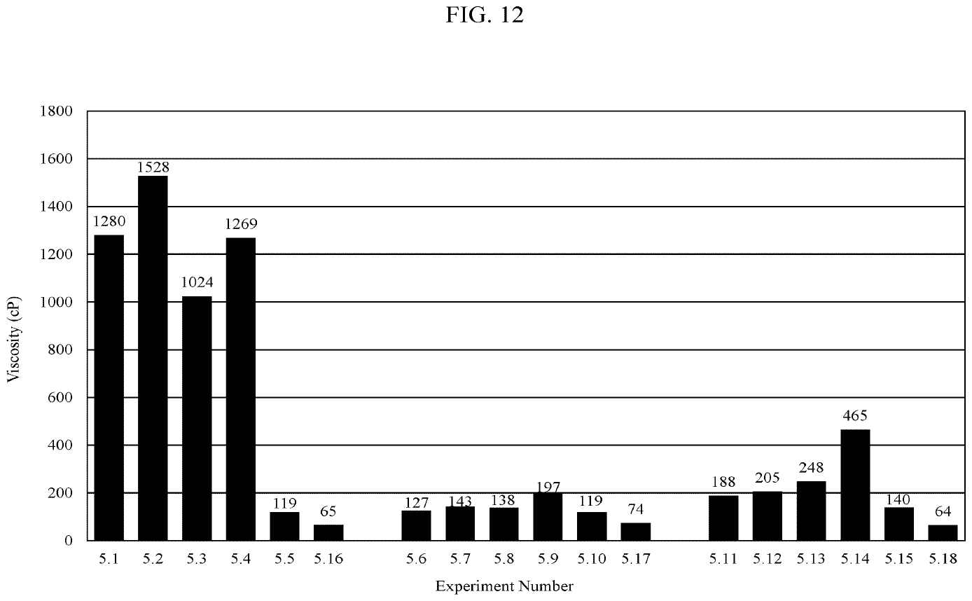

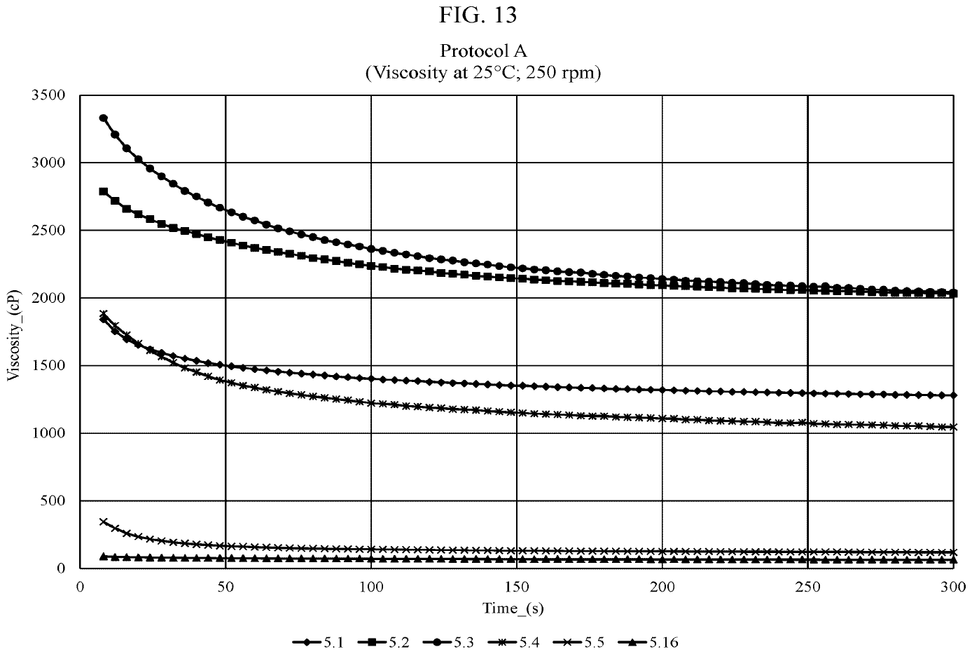

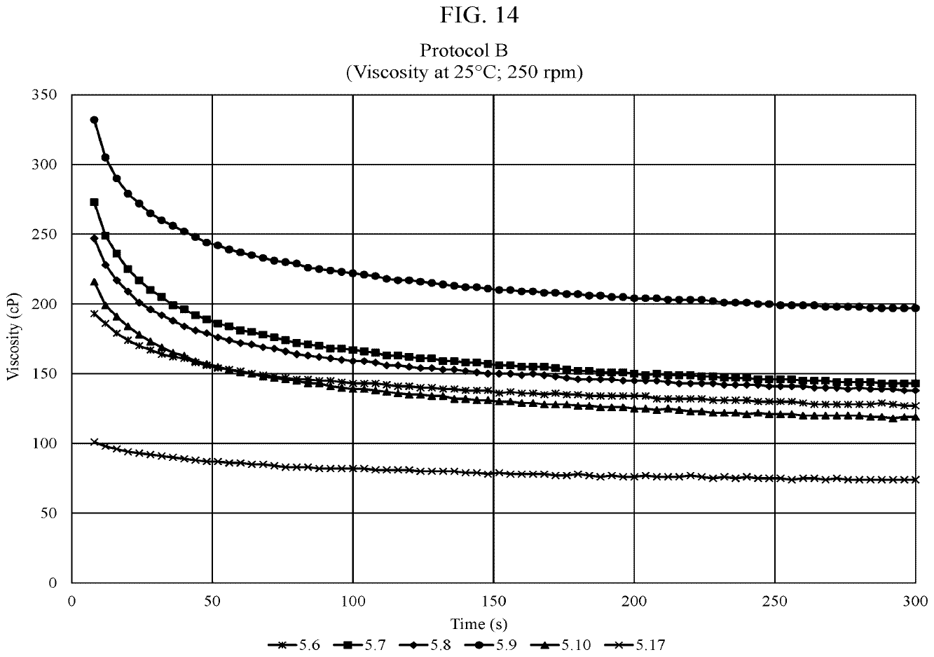

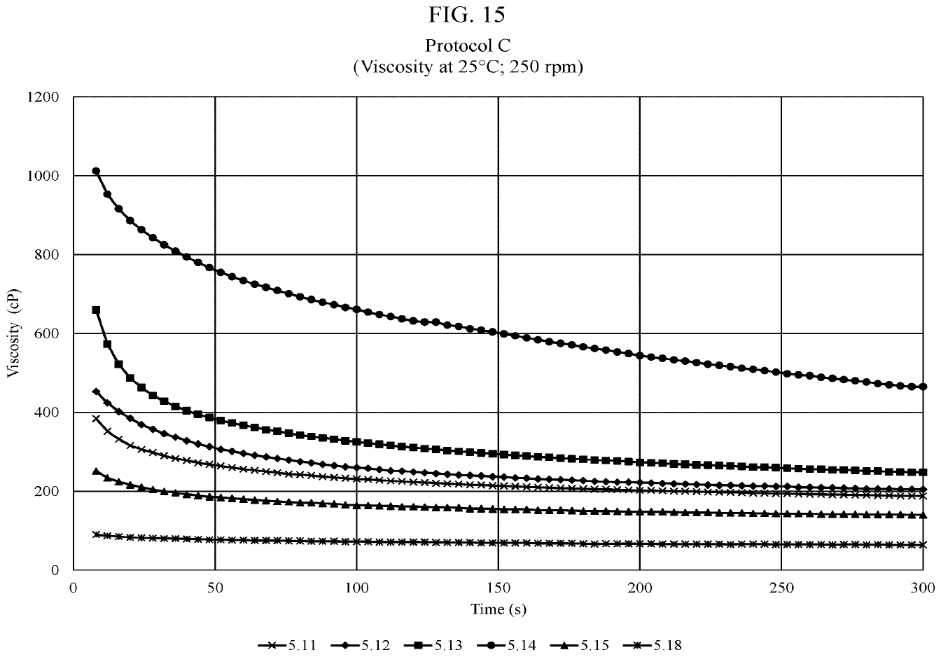

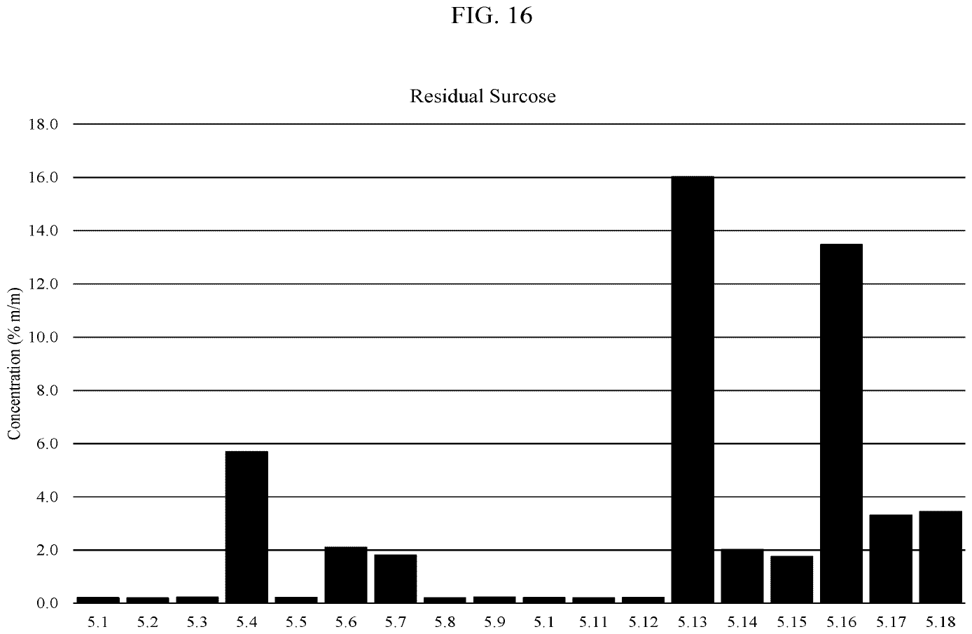

Resulting samples from the cultures outlined in Table 7 were evaluated for aspect, texture, and syneresis. Aspect was evaluated visually. Results are outlined in Tables 8 and 9 and FIGS. 12-16.

Overall, the C18X1, B3K7, C22B11, and C18X24 strains produced pea protein fermentates with higher viscosity than the YO-MIX 433 culture. In both the Example 1 experimental conditions and experimental conditions of US2020/296982, the strains C18X1, B3K7, C22B11, and C18X24 resulted in a product with higher viscosity and lower residual sucrose. When the fermentation conditions of US2020/296982 are used with the 25 °C fermentation temperature of Example 1, given the lower temperature preference of the current stains, the strains C18X1, B3K7, C22B11, and C18X24 again show higher viscosity products than the YO-MIX 433 culture. Additionally, the products of the fermentations with strains C18X1, B3K7, C22B11, and C18X24 had significantly different aspect than the product of the YO-MIX 433 culture. For example, samples 5.1-5.4 were creamy and thick or elastic and stretchy, but sample 5.5 showed high syneresis and got runny when stirred. Likewise, samples 5.11-5.14 were low syneresis gels that got creamier with stirring, while sample 5.15 had high syneresis and got runny when stirred. Although the bacterium in the YO-MIX 433 culture were characterized as lactic acid bacterium, they did not produce pea protein fermentates with creamy texture, low syneresis, and high viscosity like the L. citreum and L. pseudomesenteroides bacterium described herein.

TABLE 6

| Protocol | Conditions | Inoculation | Temperature | Time | Description |
|---|---|---|---|---|---|
| A | 250 mL water; | 2.0 wt % | 25 °C | 24 h | This protocol is based on the |
| | 4 wt % PURIS 870; | | | | fermentation protocol used in |
| | 15 wt % sucrose | | | | Example 1 |
| B | 250 mL water; | 2.0 wt % | 43 °C | 24 h | This protocol is based on the |
| | 5 wt % PURIS 870; | | | | fermentation protocol used in |
| | 3 wt % sucrose | | | | US2020/296982 |
| C | 250 mL water; | 2.0 wt % | 25 °C | 24 h | This protocol uses the temperature |
| | 5 wt % PURIS 870; | | | | from Example 1 and the pea |
| | 3 wt % sucrose | | | | protein and sucrose concentrations |
| | | | | | from US2020/296982 |

TABLE 7

| | | Fermentation | Inoculum | |
|---|---|---|---|---|
| Sample | Strain/Culture | Protocol | age? | Appearance |
| 5.1 | C18X1 | A | 4.5 | creamy, very thick |
| 5.2 | B3K7 | A | 4.5 | creamy, very thick |
| 5.3 | C22B11 | A | 4.5 | creamy, very thick |
| 5.4 | C18X24 | A | 4.5 | elastic, stretchy |
| 5.5 | YO-MIX 433 | A | fresh | gel with high syneresis; when mixed gets runny |
| 5.6 | C18X1 | B | 4.5 | gel with syneresis; when mixed gets very liquid |
| 5.7 | B3K7 | B | 4.5 | gel with syneresis; when mixed gets very liquid |
| 5.8 | C22B11 | B | 4.5 | gel with syneresis; when mixed gets very liquid |
| 5.9 | C18X24 | B | 4.5 | gel with syneresis; when mixed gets very liquid |
| 5.10 | YO-MIX 433 | B | fresh | gel with high syneresis; when mixed gets runny |
| 5.11 | C18X1 | C | 4.5 | gel with little syneresis; when mixed gets creamy |
| 5.12 | B3K7 | C | 4.5 | gel with little syneresis; when mixed gets creamy |
| 5.13 | C22B11 | C | 4.5 | gel with little syneresis; when mixed gets creamy |
| 5.14 | C18X24 | C | 4.5 | gel with little syneresis; when mixed gets creamy |
| 5.15 | YO-MIX 433 | C | fresh | gel with high syneresis; when mixed gets runny |
| 5.16 | Blank | A | 4.5 | separated; liquid |
| 5.17 | Blank | B | 4.5 | separated; liquid |
| 5.18 | Blank | C | 4.5 | separated; liquid |

TABLE 8

| Sample | pH | Viscosity (cP) |
|---|---|---|
| 5.1 | 4.1 | 1280 |
| 5.2 | 4.18 | 1528 |
| 5.3 | 4.07 | 1024 |
| 5.4 | 4.03 | 1269 |
| 5.5 | 4.56 | 119 |
| 5.6 | 4.41 | 127 |
| 5.7 | 4.72 | 143 |
| 5.8 | 4.57 | 138 |
| 5.9 | 4.58 | 197 |
| 5.10 | 4.11 | 119 |
| 5.11 | 4.43 | 188 |
| 5.12 | 4.43 | 205 |
| 5.13 | 4.32 | 248 |
| 5.14 | 4.03 | 465 |
| 5.15 | 4.4 | 140 |
| 5.16 | 6.98 | 65 |
| 5.17 | 7.1 | 74 |
| 5.18 | 7.05 | 64 |

TABLE 9

Concentration (% m/m)

| Sample | Glucose | Fructose | Sucrose | Mannitol |
|---|---|---|---|---|
| 5.1 | 0.0 | 5.8 | 0.2 | 1.4 |
| 5.2 | 0.0 | 6.3 | 0.2 | 1.2 |
| 5.3 | 0.0 | 6.0 | 0.2 | 1.4 |
| 5.4 | 0.0 | 3.4 | 5.7 | 1.5 |
| 5.5 | 0.0 | 0.4 | 0.2 | 1.2 |
| 5.6 | 0.0 | 0.1 | 2.1 | 0.6 |
| 5.7 | 0.0 | 0.0 | 1.8 | 0.6 |
| 5.8 | 0.0 | 0.8 | 0.2 | 0.5 |
| 5.9 | 0.0 | 0.2 | 0.2 | 0.7 |
| 5.10 | 0.0 | 0.0 | 0.2 | 1.0 |
| 5.11 | 0.0 | 0.1 | 0.2 | 0.4 |
| 5.12 | 0.0 | 0.0 | 0.2 | 0.8 |
| 5.13 | 0.4 | 0.7 | 16.0 | 0.0 |
| 5.14 | 0.0 | 0.1 | 2.0 | 0.0 |
| 5.15 | 0.1 | 0.2 | 1.8 | 0.0 |
| 5.16 | 0.2 | 0.5 | 13.5 | 0.0 |
| 5.17 | 0.0 | 0.5 | 3.3 | 0.0 |
| 5.18 | 0.0 | 0.0 | 3.4 | 0.0 |

### Example 6—Comparative Plant Protein Fermentation

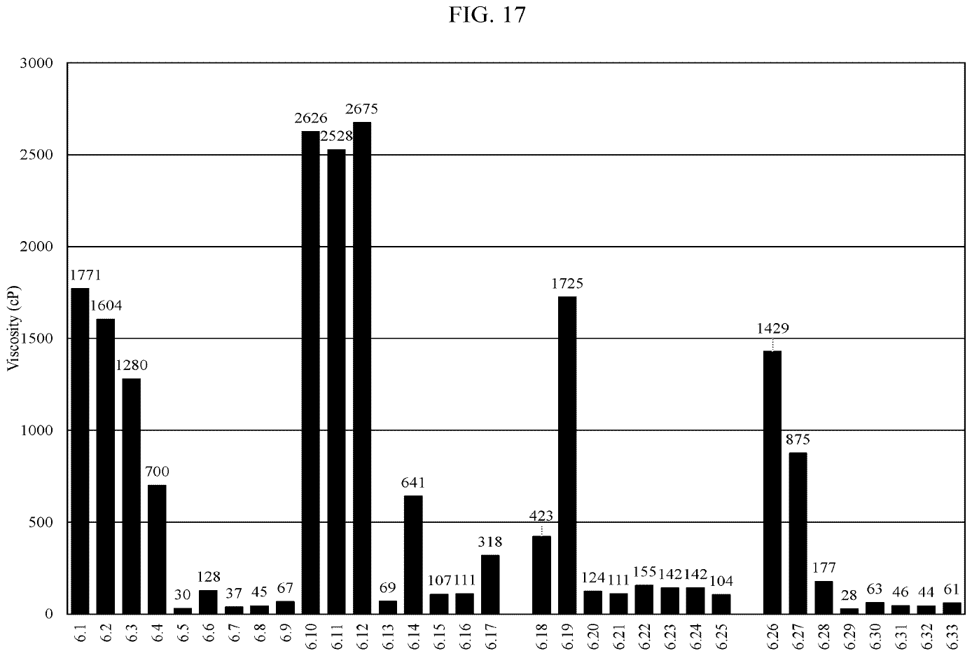

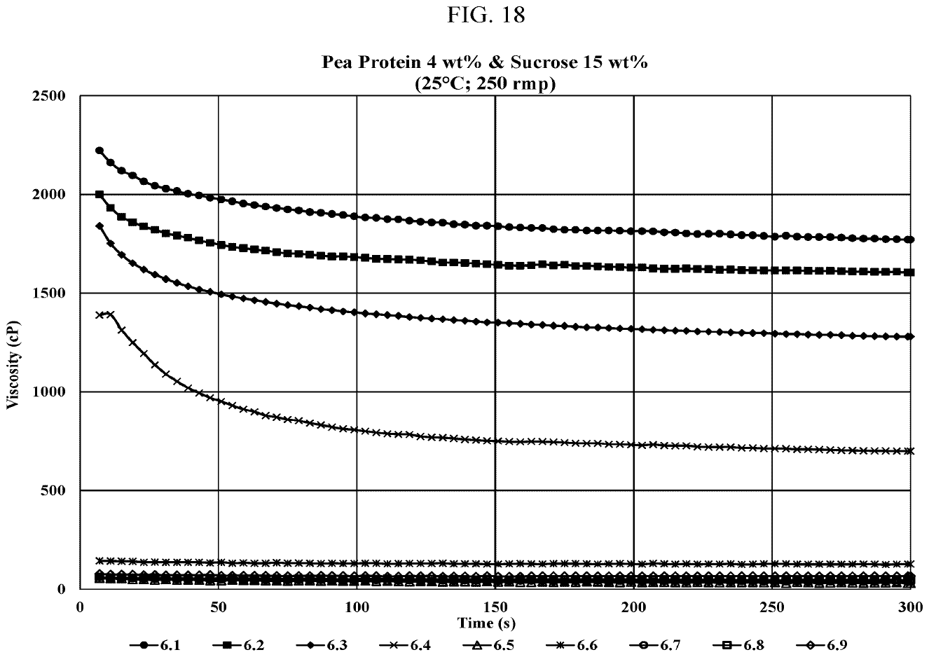

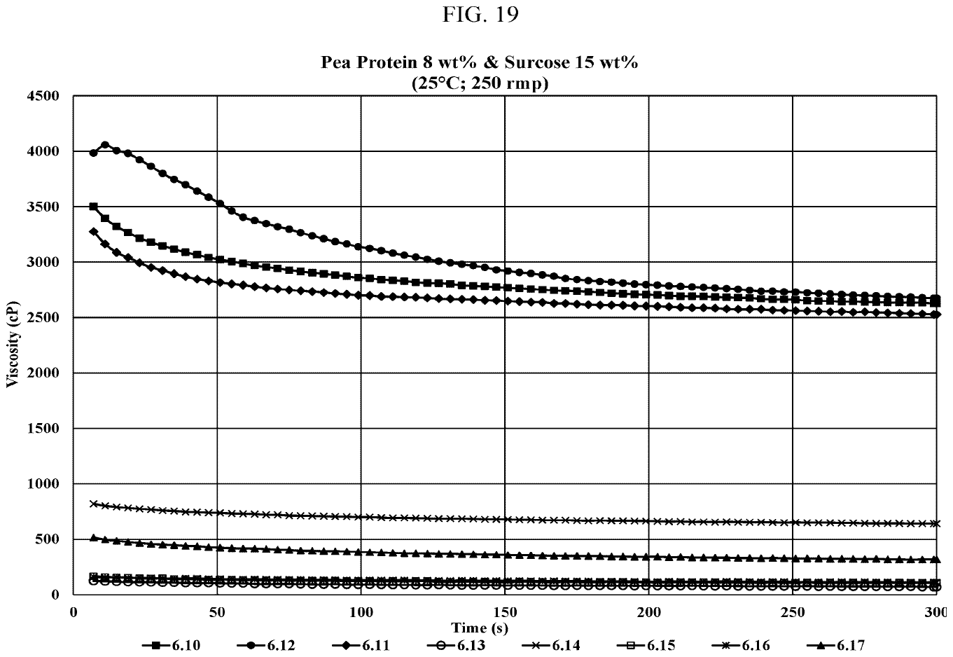

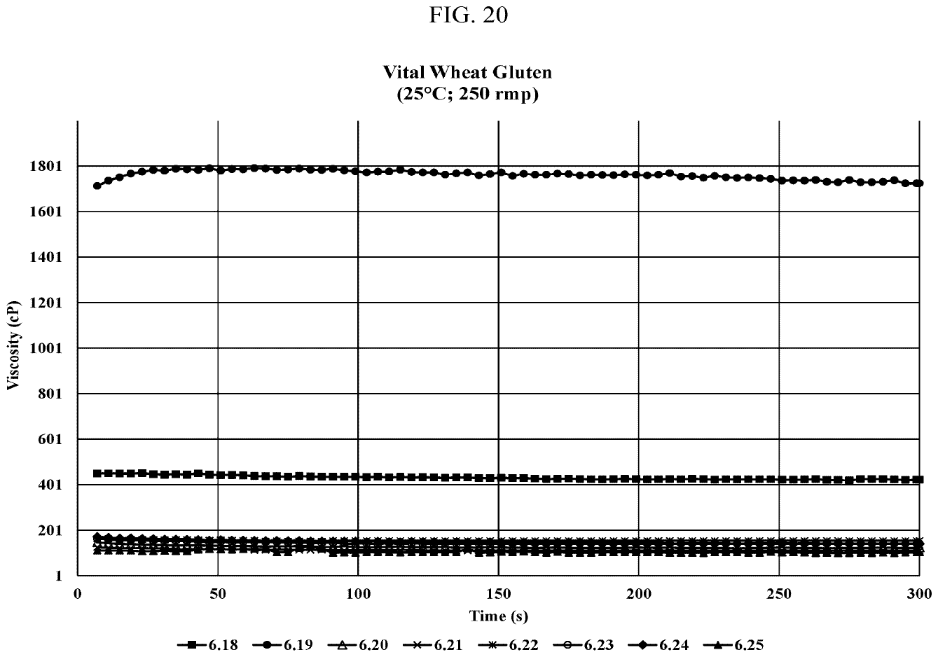

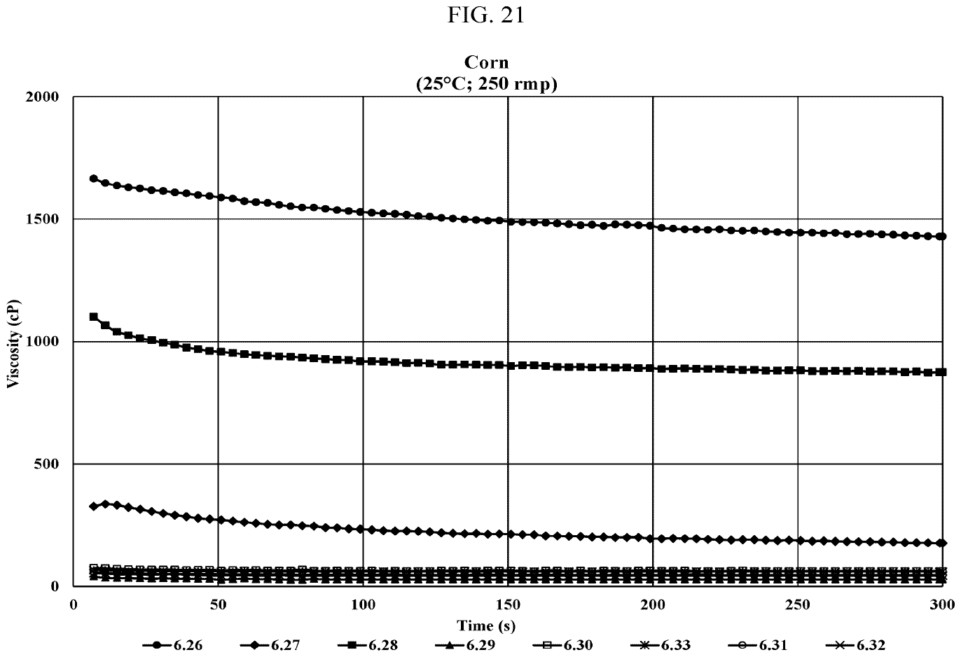

In this Example, 9 different bacterial strains are used to ferment three different plant-based proteins: pea protein, vital wheat gluten, and corn protein. In addition to the four strains described herein, the five additional publicly available strains tested are outlined in Table 10. Each fermentation condition was inoculated with 3.8 wt of the indicated strain of bacterium. Fermentations were carried out in water. The cultures outlined in Table 9 were fermented at 25 °C for 24 hours without stirring. The resulting samples were then evaluated for viscosity. Results are shown in Table 11 and FIGS. 17-21.

TABLE 10

| Species | Strain | Deposit Collection Code |
|---|---|---|
| Leuconostoc citreum | TCV-482 | NRRL B742 / ATTC 13146 |
| Leuconostoc mesenteroides | TCV-447 | NRRL B21297 |
| Leuconostoc mesenteroides | TCV-474 | NRRL B30821 |
| Leuconostoc mesenteroides | TCV-464 | NRRL B512-F |
| Leuconostoc mesenteroides | TCV-487 | NRRL B1299 / ATTC 11449 |

TABLE 11

| | | | Plant | Sucrose | Viscosity |
|---|---|---|---|---|---|
| Sample | Plant Protein | Strain | Protein wt % | wt % | (cP) |
| 6.1 | Pea Protein | B3K7 | 4 | 15 | 1771 |
| 6.2 | PURIS 870 | C22B11 | 4 | 15 | 1604 |
| 6.3 | | C18X1 | 4 | 15 | 1280 |
| 6.4 | | C18X24 | 4 | 15 | 700 |
| 6.5 | | TCV-447 | 4 | 15 | 30 |
| 6.6 | | TCV-464 | 4 | 15 | 128 |
| 6.7 | | TCV-474 | 4 | 15 | 37 |
| 6.8 | | TCV-482 | 4 | 15 | 45 |
| 6.9 | | TCV-487 | 4 | 15 | 67 |
| 6.10 | | B3K7 | 8 | 15 | 2626 |
| 6.11 | | C22B11 | 8 | 15 | 2528 |
| 6.12 | | C18X24 | 8 | 15 | 2675 |
| 6.13 | | TCV-447 | 8 | 15 | 69 |
| 6.14 | | TCV-464 | 8 | 15 | 641 |
| 6.15 | | TCV-474 | 8 | 15 | 107 |
| 6.16 | | TCV-482 | 8 | 15 | 111 |
| 6.17 | | TCV-487 | 8 | 15 | 318 |
| 6.18 | Vital Wheat | B3K7 | 8 | 15 | 423 |
| 6.19 | Gluten | C22B11 | 8 | 15 | 1725 |
| 6.20 | | C18X24 | 8 | 15 | 124 |
| 6.21 | | TCV-447 | 8 | 15 | 111 |
| 6.22 | | TCV-464 | 8 | 15 | 155 |
| 6.23 | | TCV-474 | 8 | 15 | 142 |
| 6.24 | | TCV-482 | 8 | 15 | 142 |
| 6.25 | | TCV-487 | 8 | 15 | 104 |
| 6.26 | Corn Protein | B3K7 | 8 | 15 | 1429 |
| 6.27 | | C22B11 | 8 | 15 | 875 |
| 6.28 | | C18X24 | 8 | 15 | 177 |
| 6.29 | | TCV-447 | 8 | 15 | 28 |
| 6.30 | | TCV-464 | 8 | 15 | 63 |
| 6.31 | | TCV-474 | 8 | 15 | 46 |
| 6.32 | | TCV-482 | 8 | 15 | 44 |
| 6.33 | | TCV-487 | 8 | 15 | 61 |

### CLAUSES DESCRIBING THE INVENTION

Clause 1. A vegan dessert composition comprising:
a fermented pea protein composition; wherein the fermented pea protein composition is made by contacting a pea protein with Leuconostoc citreum B3K7 (BCCM Accession No. LMG P-32801) or Leuconostoc citreum C22B11 (BCCM Accession No. LMG P-32800) in the presence of sucrose; and
optionally, a sweetener, a flavoring, or combinations thereof.

Clause 2. The composition of clause 1, wherein the vegan dessert composition is free of added starch.

Clause 3. The composition of clause 1 or clause 2, wherein the vegan dessert composition comprises a sweetener.

Clause 4. The composition of clause 3, wherein the sweetener comprises steviol glycosides, mogrosides, sucrose, fructose, glucose, erythritol, maltitol, lactitol, sorbitol, mannitol, xylitol, sucralose, acesulfame K, aspartame, saccharin, or combinations thereof.

Clause 5. A method for producing a vegan dessert composition, the method comprising:
(i) contacting a pea protein composition comprising a pea protein and sucrose with Leuconostoc citreum B3K7 (BCCM Accession No. LMG P-32801) or Leuconostoc citreum C22B11 (BCCM Accession No. LMG P-32800) for a time and under conditions sufficient to produce a fermented pea protein product;
(ii) optionally, inactivating the Leuconostoc citreum bacteria in the fermented pea protein product (e.g., by pasteurization, heat killing, irradiation, or chemical treatment) and
(iii) adding to the fermented pea protein product a sweetener, a flavoring, or combinations thereof, whereby the vegan pudding composition is produced.

Clause 6. The method of clause 5, wherein the pea protein composition comprises between 1 wt % and 20 wt %, 2 wt % and 18 wt %, or 4 wt % and 15 wt % pea protein and/or between 1 wt % and 30 wt %, 2 wt % and 25 wt %, or 5 wt % and 20 wt % sucrose.

Clause 7. The method of clause 5 or 6, wherein the pea protein composition is fermented for at least 6, at least 12, at least 18, or at least 24 hours; and/or the pea protein composition is fermented at a temperature between 20 °C and 30 °C, between 22 °C and 28 °C, between 24 °C and 26 °C, or about 25 °C.

Clause 8. The method of any one of clauses 5-7, additionally comprising the step of stirring the contacted pea protein composition during or after step (i).

Clause 9. The method of any one of clauses 5-8, wherein the sweetener comprises steviol glycosides, mogrosides, sucrose, fructose, glucose, erythritol, maltitol, lactitol, sorbitol, mannitol, xylitol, sucralose, acesulfame K, aspartame, saccharin, or combinations thereof.

Clause 10. A composition comprising Leuconostoc citreum B3K7 (BCCM Accession No. LMG P-32801), sucrose, and a pea protein.

Clause 11. Isolated Leuconostoc citreum B3K7 (BCCM Accession No. LMG P-32801) bacterial cells.

Clause 12. Use of Leuconostoc citreum B3K7 (BCCM Accession No. LMG P-32801) for the production of a vegan dessert composition.

Clause 13. A composition comprising Leuconostoc citreum C22B11 (BCCM Accession No. LMG P-32800), sucrose, and a pea protein.

Clause 14. Isolated Leuconostoc citreum C22B11 (BCCM Accession No. LMG P-32800) bacterial cells.

Clause 15. Use of Leuconostoc citreum C22B11 (BCCM Accession No. LMG P-32800) for the production of a vegan dessert composition.

Clause 16. A composition comprising:
a fermented pea protein; and
a non-viable Leuconostoc citreum B3K7 (BCCM Accession No. LMG P-32801) and/or Leuconostoc citreum C22B11 (BCCM Accession No. LMG P-32800) bacterium;
   wherein the composition is free of added sucrose and/or wherein the composition is free of added starch.

1. A vegan dessert composition comprising:
a fermented pea protein composition; wherein the fermented pea protein composition is made by contacting a pea protein with Leuconostoc citreum B3K7 (BCCM Accession No. LMG P-32801) or Leuconostoc citreum C22B11 (BCCM Accession No. LMG P-32800) in the presence of sucrose; and
a sweetener, a flavoring, or combinations thereof.

2. The composition of claim 1, wherein the vegan dessert composition is free of added starch.

3. The composition of claim 1, wherein the vegan dessert composition comprises a sweetener.

4. The composition of claim 3, wherein the sweetener comprises steviol glycosides, mogrosides, sucrose, fructose, glucose, erythritol, maltitol, lactitol, sorbitol, mannitol, xylitol, sucralose, acesulfame K, aspartame, saccharin, or combinations thereof.

5. A method for producing a vegan dessert composition, the method comprising:
(i) contacting a pea protein composition comprising a pea protein and sucrose with Leuconostoc citreum B3K7 (BCCM Accession No. LMG P-32801) or Leuconostoc citreum C22B11 (BCCM Accession No. LMG P-32800) for a time and under conditions sufficient to produce a fermented pea protein product;
(ii) optionally, inactivating the Leuconostoc citreum bacteria in the fermented pea protein product and
(ii) adding to the fermented pea protein product a sweetener, a flavoring, or combinations thereof, whereby the vegan dessert composition is produced.

6. The method of claim 5, wherein the pea protein composition comprises between 1 wt % and 20 wt % pea protein and between 1 wt % and 30 wt % sucrose.

7. The method of claim 5, wherein the pea protein composition is fermented for at least 6 hours.

8. The method of claim 5, additionally comprising the step of stirring the contacted pea protein composition during or after step (i).

9. The method of claim 5, wherein the sweetener comprises steviol glycosides, mogrosides, sucrose, fructose, glucose, erythritol, maltitol, lactitol, sorbitol, mannitol, xylitol, sucralose, acesulfame K, aspartame, saccharin, or combinations thereof.

10. A composition comprising Leuconostoc citreum B3K7 (BCCM Accession No. LMG P-32801) or Leuconostoc citreum C22B11 (BCCM Accession No. LMG P-32800), sucrose, and a pea protein.

11.-15. (canceled)

16. The method of claim 5, wherein the bacterium is inactivated by pasteurization, heat killing, irradiation, or chemical treatment.

17. The method of claim 5, wherein the pea protein composition is fermented at a temperature between 20 °C and 30 °C.

18. The method of claim 5, wherein the pea protein composition comprises 4 wt % to 15 wt % pea protein and 5 wt % to 20 wt % sucrose.
